SEQUENCE LISTING (15 sequences)

SEQ ID NO: 1 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 20 aa, Homo sapiens
MALWMRLLPLLALLALWGPD

SEQ ID NO: 3 - protein, 20 aa, Homo sapiens
LALLALWGPDPAAAFVNQHL

SEQ ID NO: 4 - protein, 20 aa, Homo sapiens
PAAAFVNQHLCGSHLVEALY

SEQ ID NO: 5 - protein, 20 aa, Homo sapiens
CGSHLVEALYLVCGERGFFY

SEQ ID NO: 6 - protein, 20 aa, Homo sapiens
LVCGERGFFYTPKTRREAED

SEQ ID NO: 7 - protein, 20 aa, Homo sapiens
TPKTRREAEDLQVGQVELGG

SEQ ID NO: 8 - protein, 20 aa, Homo sapiens
LQVGQVELGGGPGAGSLQPL

SEQ ID NO: 9 - protein, 20 aa, Homo sapiens
GPGAGSLQPLALEGSLQKRG

SEQ ID NO: 10 - protein, 20 aa, Homo sapiens
ALEGSLQKRGIVEQCCTSIC

SEQ ID NO: 11 - protein, 20 aa, Homo sapiens
IVEQCCTSICSLYQLENYCN

SEQ ID NO: 12 - protein, 24 aa, Homo sapiens
MALWMRLLPLLALLALWGPDPAAA

SEQ ID NO: 13 - protein, 30 aa, Homo sapiens
FVNQHLCGSHLVEALYLVCGERGFFYTPKT

SEQ ID NO: 14 - protein, 31 aa, Homo sapiens
EAEDLQVGQVELGGGPGAGSLQPLALEGSLQ

SEQ ID NO: 15 - protein, 21 aa, Homo sapiens
GIVEQCCTSICSLYQLENYCN

(19) United States
(12) Patent Application Publication
(10) Pub. No.: US 2024/0277768 A1
(43) Pub. Date: Aug. 22, 2024

(54) Individualized Cell Therapy Using Patient-Derived Antigen-Specific Regulatory T Cells

(71) Applicants: Tihamer ORBAN, London (GB); Jalahej HEYMAN, London (GB); Nara DAUBENEY, London (GB); Piers DAUNENEY, London (GB); PHAIN PHARMA LTD., London (GB)

(72) Inventors: Tihamer ORBAN, Brookline, MA (US); Jalahej HEYMAN, London (GB); Nara DAUBENEY, London (GB); Piers DAUBENEY, London (GB)

(21) Appl. No.: 18/571,313

(22) PCT Filed: Jun. 16, 2022
(86) PCT No.: PCT/IB2022/055610

§ 371 Date: Dec. 18, 2023

Related U.S. Application Data
(60) Provisional application No. 63/211,761, filed on Jun. 17, 2021.

(51) Int. Cl. A61K 35/17; A61K 38/28; A61K 39/00; C12N 5/0783; G01N 21/64; G01N 33/569

(52) U.S. Cl. CPC A61K 35/17; A61K 38/28; A61K 39/4611; A61K 39/464444; C12N 5/0637; G01N 21/6486; G01N 33/56972; G01N 2800/042; G01N 2800/52

(57) ABSTRACT

Compositions comprising one or more peptide fragments of preproinsulin for treating type 1 diabetes mellitus (T1DM) autoimmunity, methods of treating T1DM autoimmunity using such compositions, and kits comprising such compositions for diagnosing and/or treating TIDM autoimmunity.

### TECHNICAL FIELD OF THE INVENTION

The present disclosure relates generally to the field of autoimmune disease and specifically to the treatment, prevention, or delayed progression of type 1 diabetes mellitus (TIDM). The present disclosure relates more particularly to immunomodulatory therapy for (TIDM) autoimmunity.

### BACKGROUND OF THE INVENTION

The onset of human type 1 diabetes mellitus (T1DM) is the clinical manifestation of β-cell failure caused by T cell mediated autoimmune destruction. T1DM results in a lifelong dependence on daily insulin injections and exposure to both the acute and late complications. Despite the significant progress that has been made in its treatment, T1DM represents a severe burden on the individual and on society. T1DM is a particular burden to children and their families, representing one of the most severe chronic childhood diseases. While the onset of T1DM can occur in adulthood, it is largely a problem in children and youngsters. There is a bimodal peak age of T1DM onset, between ages 4-7 and ages 14-16 years. The worldwide incidence of T1DM is increasing, with the greatest increase in children under the age of 5 years. Therefore, there is an urgent and growing need to ameliorate this disease.

T1DM is a common endocrine disease in children, and up to 80% of children with T1DM also have diabetic ketoacidosis (DKA), which is associated with both short-term risks and long-term consequences. Short-term, and often life threatening, complications include hypo and hyperglycemic episodes often complicated with acidosis. Long-term complications can represent further significant morbidity and mortality. Patients may face both macro and microvascular complications, cardiovascular complications, hypertension, retinopathy, nephropathy, and neuropathy, which can be debilitating and life threatening. These can be reduced with improved care, but currentlycannot be eliminated in T1DM patients. Further severe complications include kidney failure, blindness, and amputation.

Despite the significant progress that has been made in its treatment, autoimmune-associated diabetes places a severe burden on affected individuals as well as on society. Insulin-dependent T1DM is an autoimmune disease, in which insulitis leads to the destruction of pancreatic β-cells. At the time of clinical onset of T1DM, significant numbers of insulin producing β-cells are destroyed, leaving only about 15% to 40% still capable of insulin production (McCulloch et al., Diabetes, 40:673-679 (1991)). This β-cell failure results in a life-long dependence on daily insulin injections and development of acute and late complications of the disease. During the natural history of the disease, the remaining functional population of β-cells inevitably dies, rendering patients dependent on exogenous insulin for life. The arrest or even the slowing of further destruction of β-cells is thus an unmet need in the field, the accomplishment of which would lead to prolonged remission and delay diabetes-related complications.

### BRIEF SUMMARY OF THE INVENTION

The present disclosure, in various aspects and embodiments, provides immunomodulatory therapy for type 1 diabetes mellitus (T1DM), including therapeutics, therapies, diagnostics, kits, and methods for making the same. For example, the disclosure provides compositions comprising a therapeutically effective amount of one or more peptide fragments of preproinsulin. The compositions can be used for treating T1DM. In addition to being immunomodulatory (e.g., as opposed to immunosuppressive), certain therapeutics in accordance with the present disclosure are not metabolically active (e.g., without insulin-like activity) and are, thus, advantageously safe for use (i.e., a large dose would not kill or harm a patient, as might a large dose of insulin).

In addition to mitigating clinical T1DM, the disclosed methods and compositions can, in certain embodiments, prevent the development or progression of pre-clinical T1DM. This can be advantageous because, in various aspects and embodiments, the disclosed methods and compositions can delay the clinical onset of T1DM, thus providing a longer symptom free period, or prevent the clinical onset of T1DM altogether. At the time of diagnosis, a T1DM patient may still have appreciable amounts of insulin production (e.g., functioning beta cells as measured by C-peptide levels). An intervention that can stop or delay the loss of functional residual beta cell mass in T1DM is highly desirable because it may provide a longer ‘remission’ period after the onset of T1DM. Furthermore, the disclosed methods and compositions may reduce or delay development of acute and chronic complications in certain patients.

Similarly, the disclosed methods and compositions may significantly improve the day-to-day management for subjects with diabetes. For example, protection against hypoglycemia and provide improved metabolic control may be provided, resulting in a delay and/or reduction in the micro and macro-vascular complications of diabetes. In summary, preservation of residual beta cell function is highly desirable as it may lead to reduction of the short- and long-term complications of T1DM.

In a first aspect, disclosed herein is a composition comprising a therapeutically effective amount of one or more peptide fragments of preproinsulin.

In some embodiments of the aforementioned aspect, the one or more peptide fragments span at least 85%, 90%, 95%, or 99% of SEQ ID NO: 1. In some instances, the one or more peptide fragments span the entire length of SEQ ID NO: 1. In certain instances, the spanned length is uninterrupted.

In some embodiments, each of the one or more peptide fragments is 10 to 30 amino acids in length. In certain instances, each of the one or more peptide fragments is 20 amino acids in length.

In some embodiments of the above aspect, each of the one or more peptide fragments comprises an amino acid sequence having at least 85% sequence identity to the amino acid sequence of any one of SEQ ID NOs: 2-11. In certain instances, each of the one or more peptide fragments comprises the amino acid sequence of any one of SEQ ID NOs: 2-11.

In some embodiments, each of the one or more peptide fragments comprises a preproinsulin epitope. In some instances, the preproinsulin epitope is not present in insulin. In certain instances, the preproinsulin epitope is not solvent accessible in insulin but is solvent accessible in preproinsulin. In some embodiments, the one or more peptide fragments do not exhibit insulin-like metabolic activity.

In some embodiments of the above aspect, the composition comprises 2, 3, 4, 5, 6, 7, 8, 9, or 10 peptide fragments. In some such instances, each peptide fragment overlaps another of the peptide fragments. In certain instances, a length of each overlap is between 5-20 amino acids. In particular instances, the length of each overlap is 10 amino acids. In some instances, each peptide fragment comprises an identical length and an identical length of overlap with a proximate peptide fragment.

In some embodiments of the above aspect, the composition disclosed herein further comprises an alum adjuvant or other pharmaceutically acceptable carrier. In some embodiments, the composition further comprises an adjuvant that promotes regulatory immune response. In some embodiments, the composition further comprises an adjuvant that includes an oil and an emulsifier. In some instances, the composition further comprises an incomplete Freund's adjuvant (IFA). In certain embodiments, the composition is immunomodulatory. In additional embodiments, the composition is not immunosuppressive. In certain instances, the composition elicits a Th2 immune response. In additional instances, the composition does not elicit a Th1 immune response.

In another aspect, provided herein is a method of treating type 1 diabetes mellitus (T1DM) autoimmunity in a subject in need thereof, by: (i) administering to the subject a composition described hereinabove in an amount sufficient to generate a response that comprises generation and/or expansion of regulatory T (Treg) cells specific to the one or more peptide fragments in the composition.

In some embodiments, the method further comprises: (ii) harvesting a population of Treg cells from the subject, wherein the population comprises Treg cells specific to the one or more peptide fragments; (iii) expanding in vitro the population of Treg cells, wherein the population comprises Treg cells specific to the one or more peptide fragments; and/or (iv) administering the expanded population of Treg cells to the subject, wherein the population comprises Treg cells specific to the one or more peptide fragments. In some such embodiments, expanding the population of Treg cells in step (iii) comprises exposing the cells to the one or more peptide fragments in vitro.

In some embodiments, the method further comprises the steps of: extracting a biological sample (e.g., blood) from the subject: measuring a level of CD4 naïve T-cells and/or a level of CD4 central memory T-cells in the sample; determining the ratio of CD4 naïve T-cell to central memory T-cell subpopulation in the sample; and administering the composition from step (i) and/or the expanded population of Treg cells from step (iv) to the subject if the ratio of CD4 naïve T-cell to central memory T-cell subpopulation is low, and/or if the level of CD4 central memory T-cell is high. In certain embodiments, the CD4 naïve T-cell is CD45RO-CD62L+ and/or the CD4 central memory T-cell is CD45RO+CD62L+. In some such embodiments, the level of CD4 naïve T-cells and/or CD4 central memory T-cells is determined by immunofluorescence analysis (e.g., flow cytometry) of the sample. In certain embodiments, the level of CD4 naïve T-cells and/or level of CD4 central memory T-cells is measured one day, one week, one month, or one year after administration of the composition in step (i) and/or after administration of the expanded population of Treg cells in step (iv). In certain embodiments, the level of CD4 naïve T-cells and/or level of CD4 central memory T-cells is measured daily, weekly, monthly, or yearly.

In some embodiments, the method further comprises the steps of: extracting a biological sample (e.g., blood) from the subject; measuring the level and/or function of Treg cells in the sample; and administering the composition from step (i) and/or the expanded population of Treg cells from step (iv) to the subject if the level and/or function of the Treg cells is low in the sample. In some such embodiments, the Treg cell is a CD4+, CD25 high, CR45RO+, Foxp3+, CD127 low, GITR+ cell. In certain instances, the function of Treg cells is measured by measuring a tolerogenic response induced by the Treg cells in the subject. In some such instances, the tolerogenic response comprises: increased expression of one or more anti-inflammatory cytokines; increased expression of one or more immunomodulatory mediators; increased expression of one or more death receptors; increased expression of indoleamine 2,3-dioxygenase (IDO) and/or heme oxygenase-1 (HO-1); and/or decreased expression of one or more pro-inflammatory cytokines. In particular instances, the anti-inflammatory cytokine compromises one or more of interleukin (IL)-1 receptor antagonist, IL-4, IL-6, IL-10, IL-11, IL-13, and transforming growth factor-β (TGF-β); the immunomodulatory mediator compromises one or more of programmed death ligand (PDL)-1/-2, cytotoxic T-lymphocyte-associated protein 4 (CTLA-4), and immunoglobulin-like transcript (ILT)-3/4; the death receptor comprises one or more of Fas, tumor necrosis factor alpha receptor (TNFαR), DR3, DR4, and DR5; and/or the pro-inflammatory cytokine comprises one or more of IL-1, IL-12, IL-18, tumor necrosis factor alpha (TNF-α), interferon gamma (IFNγ), and granulocyte-macrophage colony stimulating factor (GM-CSF). In some embodiments, the level and/or function of Treg cells is determined by immunofluorescence analysis (e.g., flow cytometry) of the sample. In certain embodiments, the level and/or function of Treg cells is measured one day, one week, one month, or one year after administration of the composition in step (i) and/or after administration of the expanded population of Treg cells in step (iv). In certain embodiments, the level and/or function of Treg cells is measured daily, weekly, monthly, or yearly.

In some embodiments, the method further comprises the steps of: extracting a biological sample (e.g., blood) from the subject; using the sample to determine pancreatic beta cell function of the subject; and administering the composition from step (i) and/or the expanded population of Treg cells from step (iv) to the subject if the pancreatic beta cell function is declining and/or starting to decline. In some such embodiments, the pancreatic beta cell function is determined by C-peptide test, such as by average C-peptide plasma concentration (CPAVE) test. In certain embodiments, the pancreatic beta cell function is determined one day, one week, one month, or one year after administration of the composition in step (i) and/or after administration of the expanded population of Treg cells in step (iv). In certain embodiments, the pancreatic beta cell function is determined daily, weekly, monthly, or yearly.

In some embodiments, a method of treatment disclosed hereinabove further comprises administering at least one proregulatory leukotriene or cytokine; and/or at least one anti-inflammatory leukotriene or cytokine. In certain embodiments, the method further comprises administering one or more beta cell promoting agents, anti-inflammatory agents, and/or anti-autoimmunity agents.

In some embodiments, the subject has T1DM and the method achieves at least one clinical endpoint. In certain embodiments, the subject has T1DM and the method mitigates at least one symptom of T1DM. In other embodiments, the subject has pre-clinical T1DM and the method prevents or delays progression to clinical T1DM. In yet other embodiments, the subject is predisposed to developing T1DM and the method prevents or delays development of T1DM.

In some embodiments, the method disclosed hereinabove mitigates autoimmunity to pancreatic beta cells.

In some embodiments of the method disclosed hereinabove, the administering step comprises intravenous, intramuscular, or subcutaneous administration.

In some embodiments, the subject is a human, such as a human adult or a human juvenile.

In another aspect, provided herein is a kit for treating T1DM autoimmunity comprising: a therapeutically effective amount of a composition disclosed hereinabove; and instructions for administration of the composition to a subject in need thereof.

In another aspect, provided herein is a kit for diagnosing and treating T1DM autoimmunity comprising: a T1DM autoimmunity diagnostic; a therapeutically effective amount of a composition disclosed hereinabove; and instructions for diagnosing a subject and administering the composition to the subject if the subject is in need thereof.

### DETAILED DESCRIPTION OF THE INVENTION

The present disclosure, in various aspects and embodiments, provides immunomodulatory therapy for type 1 diabetes mellitus (T1DM) autoimmunity, including therapeutics, therapies, kits, and methods for making the same. For example, the disclosure provides compositions for treating T1DM autoimmunity comprising a therapeutically effective amount of one or more peptide fragments of preproinsulin.

### References and Definitions

All publications, patent applications, patents, and other references mentioned herein are incorporated by reference herein in their entirety. The patent and scientific literature referred to herein establishes knowledge that is available to those of skill in the art. The issued US patents, allowed applications, published foreign applications, and references, which are cited herein are hereby incorporated by reference in their entirety to the same extent as if each was specifically and individually indicated to be incorporated by reference.

Aspects of the present disclosure can be embodied in different forms and should not be construed as limited to the embodiments set forth herein. Rather, these embodiments are provided so that this disclosure will be thorough and complete, and will fully convey the scope of the disclosure to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant disclosure.

Unless otherwise defined, all technical and scientific terms used herein have the same meaning as commonly understood by one of ordinary skill in the art to which this disclosure relates. The terminology used in the description herein is for the purpose of describing particular embodiments only and is not intended to be limiting unless clearly indicated otherwise by context.

As used in the specification and the appended claims, the singular forms “a,” “an” and “the” include plural referents unless the context clearly dictates otherwise. Thus, for example, reference to “a peptide fragment of preproinsulin,” “a composition” or “an additional therapeutic” can include mixtures of two or more such peptide fragment of preproinsulin, composition, or additional therapeutics, and the like.

As used herein, unless specifically indicated otherwise, the word “or” is used in the inclusive sense of “and/or” and not the exclusive sense of “either/or.” The term “and/or” encompasses embodiments in which both or either of the linked features are true or present.

The term “about” or “approximately” generally means within 10%, preferably within 5%, or more preferably within 1%, of a given value or range, unless dictated otherwise by context.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. In a particular aspect, the subject is human.

A “patient” refers to a subject who shows symptoms and/or complications of type 1 diabetes mellitus (T1DM), is diagnosed with T1DM, is under the treatment of a clinician, e.g., physician for T1DM, has pre-clinical T1DM, and/or is at a risk of developing T1DM. The term “patient” includes human and veterinary subjects. Any reference to subjects in the present disclosure, should be understood to include the possibility that the subject is a “patient” unless clearly dictated otherwise by context.

As used herein, the term “treatment” refers to the medical management of a subject, such as a patient, with the intent to cure, ameliorate, stabilize, or prevent type 1 diabetes mellitus (T1DM). This term includes active treatment (treatment directed to improve T1DM), causal treatment (treatment directed to the cause of T1DM), palliative treatment (treatment designed for the relief of symptoms or complications associated with T1DM), preventative treatment (treatment directed to delaying, minimizing, or partially or completely inhibiting the development or onset of T1DM); and supportive treatment (treatment employed to supplement another therapy). Treatment also includes curing, suppressing, reducing, alleviating, and/or ameliorating one or more symptoms and/or complications associated with T1DM. In some embodiments, treatment can include achieving at least one clinical endpoint of T1DM, such as improved C-peptide secretion, reduced insulin use, improved HbAlc, closer to normal blood sugar levels, less blood sugar level fluctuation, and the like. In some embodiments, treatment can include reducing or mitigating at least one symptom of T1DM.

For example, treatment can include reducing the frequency of hypoglycemia/hyperglycemia, reducing glucosuria, reducing a level/number of hospitalization(s), and reducing a level/number of complications such as nephropathy, neuropathy, and retinopathy. In particular, treatment can include reducing at least one symptom of T1DM by at least 5%, such as, 5%, 10%, 15%, 20%, 25%, 30%, 35%, 40%, 45%, 50%, 55%, 60%, 65%, 70%, 75%, 80%, 85%, 90%, 95%, 99% or more, as determined relative to a suitable control. A suitable control may be a similar symptom in a control subject, such as a test subject before receiving the treatment method described herein, or a different subject or group of subjects with like symptoms as the test subject, who did not receive the treatment described herein.

Treatment can also include prevention and/or delay of the onset of symptoms and/or complications associated with T1DM. Treatment also includes diminishment of the extent of T1DM; delaying or slowing the progress of the T1DM; preventing, delaying or slowing the progress of pre-clinical T1DM to clinical T1DM; preventing, delaying or slowing development of T1DM in a subject who is at a risk of developing T1DM; amelioration or palliation of T1DM; and remission (whether partial or total), whether detectable or undetectable.

“Ameliorating” or “palliating” T1DM means that the extent and/or undesirable clinical manifestations of T1DM are lessened and/or the time course of the progression is slowed or lengthened, as compared to the extent or time course in the absence of treatment. Treatment does not require the complete amelioration of a symptom, complication, or disease and encompasses embodiments in which one reduces symptoms and/or underlying risk factors.

“Treatment” can also mean prolonging survival as compared to expected survival if not receiving treatment. Those in need of treatment include those already with T1DM, as well as those prone to have the condition or disorder or those in which the condition or disorder is to be prevented. The term “prevent” does not require the 100% elimination of the possibility of an event. Rather, it denotes that the likelihood of the occurrence of the event has been reduced in the presence of the compound or method. In various aspects, the term covers any treatment of a subject, including a mammal (e.g., a human), and includes: (i) preventing T1DM from occurring in a subject that can be predisposed to the disease but has not yet been diagnosed as having it; (ii) inhibiting T1DM, such as arresting its development; or (iii) relieving T1DM, such as causing regression of the T1DM.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compositions or methods disclosed herein. In some aspects of the disclosed methods, the subject has been diagnosed with a need for treatment of T1DM prior to the administering step. As used herein, a subject in need of a treatment may refer to identification of or selection of a subject based upon need for treatment of T1DM. It is contemplated that the identification can, in one aspect, be performed by a person different from the person making the diagnosis. It is also contemplated, in a further aspect, that the administration can be performed by one who previously made the diagnosis.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intra-aural administration, intracerebral administration, rectal administration, and parenteral administration, including injectable administration such as intravenous administration, intra-arterial administration, intramuscular administration, and subcutaneous administration. Administration can be continuous or intermittent. In various aspects, a preparation can be administered therapeutically, such as administered to treat an existing disease or condition, such as T1DM. In further aspects, a preparation can be administered prophylactically, such as administered for prevention of a disease or condition, such as T1DM.

As used herein, the term “effective amount” or “amount effective” or “therapeutically effective amount” refer to an amount that is sufficient to achieve the desired result or to have an effect on an undesired condition. For example, a “therapeutically effective amount” may refer to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

For example, it is well within the skill of the art to start doses of a therapeutic at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a prophylactically effective amount, such as an amount effective for prevention of a disease or condition, such as T1DM.

In some embodiments, a therapeutically effective amount of preproinsulin peptide fragments can be 5 micrograms to 10 milligrams of preproinsulin peptide fragments, 0.5 to 4.0 milligrams of preproinsulin peptide fragments, or any value there between. In some embodiments, a therapeutically effective amount of preproinsulin peptide fragments can be 5, 10, 25, 50, 75, 100, 125, 150, 175, 200, 225, 250, 275, 300, 350, 400, 450, 500, 600, 700, 800, or 900 micrograms of preproinsulin peptide fragments, or any value there between. In some embodiments, a therapeutically effective amount of preproinsulin peptide fragments can be 1.0, 1.25, 1.5, 1.75, 2.0, 2.25, 2.5, 2.75, 3.0, 3.25, 3.5, 3.75, 4.0, 4.25, 4.5, 4.75, 5.0, 5.5, 6.0, 6.5, 7.0, 7.5, 8.0, 8.5, 9.0, 9.5, or 10 milligrams of preproinsulin peptide fragments, or any value there between. Additionally or alternatively, a therapeutically effective amount of preproinsulin peptide fragments may be an amount that can elicit a desirable immune response in a subject (e.g., a desirable level of antigen-specific Treg cells, suppression of cytotoxic T cell function, generation of a tolerogenic response, generation of a Th2/Treg response, etc.). In further or alternative instances, a therapeutically effective amount of preproinsulin peptide fragments is an amount that can achieve at least one clinical endpoint (e.g., improved C-peptide secretion, reduced insulin use, improved HbAlc, closer to normal blood sugar levels, less blood sugar level fluctuation, and the like) in a subject. Additionally or alternatively, a therapeutically effective amount of preproinsulin peptide fragments may be an amount that can mitigate at least one symptom of the T1DM (e.g., frequency of hypoglycemia/hyperglycemia, reduced glucosuria, level/number of hospitalization(s), and level/number of complications such as nephropathy, neuropathy, and retinopathy).

As used herein with reference to preproinsulin peptide fragments or a composition containing the same, the term “unit dosage form” refers to the amount of the one or more preproinsulin peptide fragments and/or the composition that is suitable for administration to a subject in a single dose. In some embodiments, a unit dosage form of one or more preproinsulin peptide fragments and/or a composition (e.g., a pharmaceutical composition) described herein may encompass a therapeutically effective amount of the preproinsulin peptide fragments and/or the composition.

The term “pharmaceutically acceptable” describes a material that is not biologically or otherwise undesirable, such as without causing an unacceptable level of undesirable biological effects or interacting in a deleterious manner.

As used herein with respect to a parameter, the term “reduce” or “reducing” or “decrease” or “decreasing” or “alleviate” or “alleviating” refers to a detectable (e.g., at least about 5%, 10%, 15%, 20%, 25%, 30%, 35%, 40%, 45%, 50%, 55%, 60%, 65%, 70%, 75%, 80%, 85%, 90%, 95%, 97%, 99%, or more) negative change in the parameter from a comparison control, e.g., an established normal or reference level of the parameter, or an established standard control. For example, as used herein, reducing or decreasing or alleviating symptoms and/or complications associated with T1DM refers to detectable (e.g., at least about 5%, 10%, 15%, 20%, 25%, 30%, 35%, 40%, 45%, 50%, 55%, 60%, 65%, 70%, 75%, 80%, 85%, 90%, 95%, 97%, 99%, or more) negative change in symptoms and/or complications associated with T1DM in a test subject (e.g., a subject who was subject to the methods of treatment described herein) compared to symptoms and/or complications associated with T1DM in a control subject (e.g., the same subject before receiving the treatment method described herein; or a different subject, or group of subjects with like symptoms as the test subject, who did not receive the treatment described herein).

As used herein, a “control” or “control subject” refers to a subject who has not received the compositions and methods of the present disclosure. As used herein, a “test subject” refers to a subject who has received the compositions and methods of the present disclosure. As used herein with reference to a parameter, a “suitable control” may refer to the parameter in a control subject (e.g., a test subject before receiving the treatment method described herein; or a different subject, or group of subjects with like symptoms as the test subject, who did not receive the treatment described herein). For example, as used herein with reference to symptoms and/or complications associated with T1DM, a “suitable control” may refer to symptoms and/or complications associated with T1DM in a control subject (e.g., a test subject before receiving the treatment method described herein; or a different subject, or group of subjects with like symptoms as the test subject, who did not receive the treatment described herein).

As used interchangeably herein, the terms “peptide fragments” or “peptide fragments of preproinsulin” or “preproinsulin peptide fragments” refer to fragments of preproinsulin protein, e.g., human preproinsulin protein. Reference to the preproinsulin peptide fragments may refer to a collection or composition of preproinsulin peptide fragments configured for use in the one of the methods or compositions described herein, such as a therapeutic composition, and specifically to the identity (e.g., sequence) of a selection of preproinsulin peptide fragments included in the composition unless dictated otherwise by context.

In some embodiments, the preproinsulin peptide fragments (within a composition) can cumulatively span at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) of the preproinsulin sequence (e.g., SEQ ID NO: 1). In some embodiments, one or more of the preproinsulin peptide fragments (optionally, each of the preproinsulin peptide fragments) can comprise an amino acid sequence having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of any one of SEQ ID NOs: 2-11. In particular embodiments, preproinsulin peptide fragments of the present disclosure can be one or more (e.g., 1, 2, 3, 4, 5, 6, 7, 8, 9, or all) of Peptide 1, Peptide 2, Peptide 3, Peptide 4, Peptide 5, Peptide 6, Peptide 7, Peptide 8, Peptide 9, and Peptide 10 described in Table 1. In some embodiments, the preproinsulin peptide fragments are overlapping fragments. For example, each peptide fragment may overlap with the peptide fragment immediately preceding or following it by about 5, 6, 7, 8, 9, 10, 11, 12, 13, 14, 15, 16, 17, 18, 19, 20, 21, 22, 23, 24, 25, 26, 27, 28, 29, or 30 amino acids. In particular embodiments, all of the peptide fragments are overlapping with at least one other peptide fragment (e.g., all but two of the peptide fragments are overlapping with at least two other peptide fragments).

As used herein, “autoantigen-specific Treg cells” may refer to Treg cells that are induced by an autoantigen and/or are specific to an autoantigen. The autoantigen of interest in the present disclosure is preproinsulin. Accordingly, as used herein, “autoantigen-specific Treg cells” may refer to preproinsulin-specific Treg cells, i.e., Treg cells that are induced by and/or are specific to one or more peptide fragments of preproinsulin or to one or more epitopes exhibited thereby.

As used herein, the term “cell therapy” refers to therapy or treatment with autoantigen-specific Treg cells. In particular, “cell therapy” refers to therapy or treatment with preproinsulin-specific Treg cells, i.e., Treg cells that are induced by and/or are specific to one or more peptide fragments of preproinsulin or to one or more epitopes exhibited thereby. For example, “cell therapy” may refer to a method of treating T1DM in a subject by administering to the subject a population of preproinsulin-specific Treg cells. Such preproinsulin-specific Treg cells can be induced, generated and/or expanded by: administration (e.g., by intravenous, intramuscular, or subcutaneous routes) of a composition comprising one or more peptide fragments of preproinsulin to a subject, in an amount sufficient to generate a response that comprises the activation, generation, and/or expansion of Treg cells specific to the one or more peptide fragments of preproinsulin or to one or more epitopes exhibited thereby; harvesting a population of Treg cells from the subject, wherein the population of Treg cells comprises Treg cells specific to the one or more peptide fragments of preproinsulin or to one or more epitopes exhibited thereby; and expansion of the population of Treg cells in vitro, wherein optionally, the expansion comprises further exposing the population of Treg cells to the one or more peptide fragments in vitro. For therapy or treatment purpose, such expanded preproinsulin-specific Treg cells can be re-introduced to the subject as and when need be.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including matters of logic with respect to arrangement of steps or operational flow; plain meaning derived from grammatical organization or punctuation; and the number or type of embodiments described in the specification.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments unless the embodiment is inoperative without those elements.

### Insulin and Preproinsulin

Autoantibodies against insulin are frequently found in newly diagnosed diabetic patients. Insulin is synthesized in the pancreatic islet β-cells from its precursor preproinsulin. Insulin is both produced and degraded within the pancreatic β-cells. Preproinsulin is a 110 amino acid biologically inactive precursor to the biologically active endocrine hormone insulin. Preproinsulin is converted into proinsulin by signal peptidases, which remove its signal peptide from its N-terminus. Finally, proinsulin is converted into the bioactive hormone insulin by removal of its connecting peptide (C-peptide).

Almost no preproinsulin exists outside β-cells because removal of the signal peptide is not a separate step, but rather is closely linked to translocation of the protein into the endoplasmic reticulum (ER). For the same reason, preproinsulin is rarely used medicinally, unlike insulin, the mature product, and proinsulin, a stable ER intermediate.

Provided herein are compositions comprising one or more peptide fragments of preproinsulin. In some embodiments, the preproinsulin is human preproinsulin (GenBank Accession No: NP_000198.1). In some embodiments, the preproinsulin is a 110 amino acid protein. The preproinsulin may comprise an amino acid sequence having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of SEQ ID NO: 1. For example, the preproinsulin may comprise the amino acid sequence of SEQ ID NO: 1. In those instances, a composition provided herein may contain one or more peptide fragments of SEQ ID NO: 1.

In some embodiments, the present disclosure contemplates not only SEQ ID NO: 1, but also homologs and analogs thereof. For example, a preproinsulin sequence disclosed herein can be structurally and/or functionally homologous to SEQ ID NO: 1. Homology can include at least 70% (e.g., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%) homology. Analogous sequences can include preproinsulin sequences from non-human species, and synthetic peptide sequences comprising one or more preproinsulin epitopes or cross-reactive epitopes. In some embodiments, analogous sequences can include human preproinsulin sequences containing one or more mutations or polymorphisms.

The first step of insulin biosynthesis involves the targeting and translocation of newly synthesized preproinsulin from the cytosol into the endoplasmic reticulum (ER). This process is led by the signal peptide of preproinsulin at its N-terminus. Preproinsulin has a 24 residue signal peptide, which comprises three regions: a positive charged n-region; a central core hydrophobic h-region; and a polar c-region containing a cleavage site of the SPase. Mutations located in the preproinsulin signal peptide that have been reported to cause diabetes include, without limitations L13R, A24D, R6C, and R6H (Liu et al., Vitam Horm, 95: 35-62 (2014); Rapoport, Nature, 450: 663-669 (2007); Liu et al., Mol Aspects Med, 42: 3-18 (2015)). The clinical diabetes phenotypes associated with these mutants range from severe neonatal-onset insulin-deficient diabetes caused by L13R or A24D, to mild adult onset diabetes associated with R6C or R6H, suggesting the possibility that different cellular defects or molecular mechanisms may underlie the onset and development of diabetes in these patients (Liu et al., Mol Aspects Med, 42: 3-18 (2015)). In some embodiments, a preproinsulin sequence of the present disclosure is a human preproinsulin sequence containing one or more of L13R, A24D, R6C, and/or R6H mutations.

Insulin biogenesis begins with the synthesis of preproinsulin in the rough ER and conversion of preproinsulin to proinsulin. Preproinsulin is converted to proinsulin shortly after (or during) translocation into the lumen of the rough ER. Proinsulin is then transported to the trans-cisternae of the Golgi complex where it is directed towards nascent, immature secretory granules. Conversion of proinsulin to insulin and C-peptide by proteolytic cleavage arises within secretory granules, and is dependent upon their acidification via ATP-dependent proton pump. The proinsulin consists of the B-chain, C-peptide and A-chain. The C-peptide is cut out and the B-chain and A-chain ends connected by disulfide bonds to form insulin. The secretory granules undergo a maturation process in which insulin content becomes crystallized with zinc and calcium as dense-core granules. These new mature dense-core insulin granules form two distinct intracellular pools, the readily releasable pools (RRP) and the reserved pool. These two populations of dense-core granules may be responsible for the biphasic nature of insulin release. The RRP granules are associated with the plasma membrane and undergo an acute calcium-dependent release responsible for first phase insulin secretion. These granule contents are discharged by exocytosis in response to an appropriate stimulus, primarily glucose. This process represents the regulated secretory pathway to which more than 99% of proinsulin is directed in beta cells of a healthy individual. In contrast, second phase insulin secretion requires the trafficking of the reserved granule pool to the plasma membrane, and involves the rapid transfer of products from the Golgi complex to the plasma membrane for immediate release. The initial trigger for insulin granule fusion with the plasma membrane is a rise in intracellular calcium and in the case of glucose stimulation results from increased production of ATP, closure of the ATP-sensitive potassium channel and cellular depolarization. In turn, this opens voltage-dependent calcium channels allowing increased influx of extracellular calcium. Calcium may bind to members of the fusion regulatory proteins synaptogamin that functionally represses the fusion inhibitory protein complex.

In brief, preproinsulin is a beta cell specific antigen and, thus, can form the basis of the immunomodulatory compositions and therapies for T1DM in accordance with the present disclosure.

### Peptide Fragments of Preproinsulin

The present disclosure, in various embodiments, utilizes preproinsulin by dividing the preproinsulin sequence, or a portion thereof, into metabolically inactive overlapping preproinsulin polypeptide fragments, to capture the immune modulatory potentials of preproinsulin as a beta cell restricted antigen. By dividing preproinsulin into overlapping peptides, the immune system can be presented with peptide sequences which comprise sequences that are unique to the preproinsulin, but that are not present either in the insulin or in the C-peptides (both of which are present in circulation).

In some embodiments, the present disclosure provides a composition comprising a therapeutically effective amount of one or more peptide fragments of preproinsulin. For example, a composition described herein may contain a therapeutically effective amount of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 peptide fragments of preproinsulin. In particular instances, a composition described herein contains a therapeutically effective amount of 10 peptide fragments of preproinsulin.

Each of the one or more peptide fragments of preproinsulin can be about 5, 6, 7, 8, 9, 10, 11, 12, 13, 14, 15, 16, 17, 18, 19, 20, 21, 22, 23, 24, 25, 26, 27, 28, 29, or 30 amino acids in length. In certain instances, each of the one or more peptide fragments of preproinsulin is about 5-10, 5-15, 5-25, 10-20, or 10-30 amino acids in length. For example, each of the one or more peptide fragments of preproinsulin can be about 20 amino acids in length. In certain instances, a composition described herein comprises two or more peptide fragments of uniform length. For example, a composition of the present disclosure may comprise one or more peptide fragments of preproinsulin, wherein each peptide fragment is 20 amino acids long. In some embodiments, compositions in accordance with the present disclosure can include fragments of uniform length (e.g., all about 20 amino acids in length) as well as distributions of different lengths. Fragment lengths, or distributions thereof, can be selected to optimize an immunomodulatory effect.

In some embodiments, the one or more preproinsulin peptide fragments can comprise an amino acid sequence having at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to the amino acid sequence of any one of SEQ ID NOs: 2-11. In certain instances, the one or more peptide fragments of preproinsulin can comprise the amino acid sequence of any one of SEQ ID NOs: 2-11. In particular, a composition of the present disclosure may comprise one or more (e.g., 1, 2, 3, 4, 5, 6, 7, 8, 9, or all) of Peptide 1, Peptide 2, Peptide 3, Peptide 4, Peptide 5, Peptide 6, Peptide 7, Peptide 8, Peptide 9, and Peptide 10 described in Table 3. Peptides 1-10 in Table 3 cumulative span the entire length of SEQ ID NO: 1, each being 20 amino acids in length, and each overlapping the preceding or following peptide (based on position within SEQ ID NO: 1), if present, by 10 amino acids.

In some embodiments, a composition described herein can be configured for treating individual subjects and/or for treating select populations of subjects. For example, for treating individual subjects and/or for treating select populations of subjects, a composition described herein may comprise one or more (e.g., 1, 2, 3, 4, 5, 6, 7, 8, 9, or all) of Peptide 1, Peptide 2, Peptide 3, Peptide 4, Peptide 5, Peptide 6, Peptide 7, Peptide 8, Peptide 9, and Peptide 10 described in Table 1. The selection of peptides (e.g., one or more (e.g., 1, 2, 3, 4, 5, 6, 7, 8, 9, or all) of Peptide 1, Peptide 2, Peptide 3, Peptide 4, Peptide 5, Peptide 6, Peptide 7, Peptide 8, Peptide 9, and Peptide 10 described in Table 1) may be based on a subject-specific profile such that the treatment is personalized to the individual or population. The selection may be based, for example, on a subject's genotype for one or more genes related to T1DM (e.g., correlated to a subject's antigen-specific autoimmune response to preproinsulin) and/or on a subject's immune response to one or more specific peptides (e.g., as measured by a stimulation assay).

In some embodiments, a composition of the present disclosure comprises overlapping peptide fragments of preproinsulin. For example, a composition described herein may comprise two or more peptide fragments, wherein each peptide fragment overlaps with another peptide fragment. In some instances, each overlap is an overlap of about 5, 6, 7, 8, 9, 10, 11, 12, 13, 14, 15, 16, 17, 18, 19, 20, 21, 22, 23, 24, 25, 26, 27, 28, 29, or 30 amino acids. In some instances, each overlap is an overlap of about 5-10, 5-15, 5-20, 5-25, or 5-30 amino acids. For example, each overlap can be an overlap of about 10 amino acids. In certain instances, a composition described herein comprises preproinsulin peptide fragments of uniform overlap (e.g., all about 10 amino acids) as well as varying overlap. Again, overlap lengths, or distributions thereof, can be selected to optimize an immunomodulatory effect.

The one or more peptide fragments described herein may span at least 75% (e.g., at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) of the preproinsulin sequence. For example, the one or more peptide fragments described herein may cumulatively span at least 75% of SEQ ID NO: 1. In some instances, the one or more peptide fragments cumulatively span the entire length of the preproinsulin sequence. Thus, a composition comprising a predetermined set of peptide fragments (e.g., one hundred fragments that are each 10 amino acids long) can encompass the entire preproinsulin sequence (e.g., 110 amino acids). In certain instances, the one or more peptide fragments span the entire length of SEQ ID NO: 1. In particular, the spanned length can be uninterrupted. In other instances, the peptide fragments do not cover the entire preproinsulin sequence, and may be limited to a set or subset of preproinsulin epitopes. The one or more peptide fragments described herein may comprise at least one internal preproinsulin epitope. An internal preproinsulin epitope is an epitope which is not normally solvent accessible in insulin. For example, an internal preproinsulin epitope may comprise an epitope which is not solvent accessible in insulin, but which is solvent accessible in preproinsulin. Additionally, an internal preproinsulin epitope may comprise an epitope which is crumpled and/or hidden inside the 3D structure of the protein but which becomes exposed during the autoimmunity process. Thus, unlike an external epitope, an internal preproinsulin epitope may not be readily available to the immune system, such as in case of an immune response that is directed against the non-denatured fully-folded protein. However, an internal preproinsulin epitope may play a major role in driving the immune response in case of autoimmunity, especially when a lot of cellular debris are produced by autoimmunity reactions.

For example, the one or more peptide fragments of preproinsulin may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more preproinsulin epitopes. In certain instances, each of the one or more peptide fragments comprises an internal preproinsulin epitope. The epitopes can be selected to optimize an immunomodulatory effect.

In some embodiments, the one or more peptide fragments of preproinsulin do not exhibit insulin-like metabolic activity (e.g., in a human subject). Such embodiments can be advantageous because they can allow for administration of concentrations of peptide fragments that are greater than a preferred, or maximum tolerated, dose of insulin. In various embodiments, the fragments comprise at least one epitope that is not present in insulin. Such embodiments can advantageously limit the effect of compositions in accordance with the present disclosure to cells containing preproinsulin.

In one embodiment, each of the overlapping peptide fragments comprises a preproinsulin epitope. Preproinsulin epitopes described herein can include known epitopes, such as B chain B9-23 and A chain 1-15 epitopes. Preproinsulin epitopes can include cryptic epitopes, which under normal conditions are not generated in sufficient amounts to be recognized by T cells undergoing deletion in thymus or anergy in the periphery. See, e.g., Lanzavecchia, Exp Med. 1995 Jun 1:181(6): 1945-8 (doi: 10.1084/jem.181.6.1945), which is herein incorporated by reference in its entirety. Cryptic preproinsulin epitopes may be exposed as a result of fragmenting preproinsulin (i.e., epitopes that are not solvent accessible in native, folded preproinsulin). During slicing and processing, following removal of the signal peptide sequence, the preproinsulin is broken up into the A chain, C peptide, and B chain. During this process, two amino acids on both ends of C peptide (i.e., four amino acids in total) are lost and not present in any further peptides. Accordingly, the border regions of the C peptide and some epitopes in that region are not expressed in insulin. However, these regions and/or epitopes are “new” and potentially immunogenic in a pathological disease setting (e.g., in the debris that are generated in the destruction process in autoimmune conditions). In some embodiments, one or more peptide fragments of the present disclosure comprises one or more of such cryptic preproinsulin epitopes. Preproinsulin epitopes can include epitopes which span the junction of the signal peptide and the B chain, the junction of the B chain and the C-peptide, or the junction of the C-peptide and the A chain, and which, therefore, are not present in insulin. Preproinsulin epitopes can include the full set of epitopes present in the preproinsulin sequence (or analog thereof). Epitopes can also include one or more epitopes that are unique to beta cells (i.e., the specific target of autoimmunity in T1DM). In some embodiments, the peptide fragments of the present disclosure comprise one or more of the insulin A-chain 1-15 epitope, the B-chain 9-23 epitope, the B-chain 11-27 epitope, the C-peptide C3-27 epitope, the C-peptide C13-32 epitope, and the C-peptide C13-20 epitope. The preproinsulin peptide fragments, compositions thereof, and methods of using or making, may be any of those described in U.S. Pat. App. Pub. No. US 2016/0361397 to Orban et al., published on Dec. 15, 2016, which is herein incorporated by reference in its entirety.

Without wishing to be bound by any particular theory, a loss of self-tolerance to insulin, a primary autoantigen, may unleash auto-aggressive T cells and initiate autoimmunity. Thus, destruction of insulin producing cells can start well before clinical onset of T1DM. At clinical diagnosis of some subjects, there can still be about 20-50% of self-insulin production, which can be completely destroyed over few years without medical intervention. The destruction process is T cell-mediated, and may involve CD4+ cells. However, regulatory T cells (Tregs) that are capable of suppressing the auto-aggressive T cell population may also play a critical role. Treg cells include naturally occurring CD4+CD25+ cells and antigen-induced CD4+Th2-like regulatory cells. An imbalance between the auto-aggressive and regulatory sets of T cells may be at the core of autoimmunity. Therefore, successful interventions may be implemented by deleting the auto-aggressive cells and/or boosting the regulatory population, in order to re-establish control and create a healthy balance.

Again, without wishing to be bound by any particular theory, antigen challenge in an autoimmune setting may stimulate beneficial changes in T cell subsets (e.g., Th2 vs. Th1), in cytokine production, and/or in induction of Treg cells. In practice, antigen-specific therapeutic approaches for autoimmune diseases may use putative self-antigens that have been implicated in the disease aetiopathogenesis. Insulin is a β-cell specific major protein and is also moderately immunogenic when used alone. However, when insulin is used, there is a concern about hypoglycemia among other side effects. Thus insulin-related peptides can be a safer choice than insulin for human use because they do not necessarily have a hypoglycemic effect. In some embodiments, a composition of the present disclosure comprises one or more peptide fragments that do not have a hypoglycemic effect when administered to a subject (e.g., a human).

Prolonged peripheral presentation of self-antigens can cause low-avidity auto reactive T cells to differentiate into memory-like auto regulatory T cells that suppress both auto reactive cytotoxic T lymphocytes (CTLs) and the presentation of self-antigens, thus, protecting beta cells from further damage. The autoimmune process in T1DM selectively kills the beta cells in the pancreatic islets and do not destroy other endocrine cells like glucagon producing alpha cells. This selectivity indicates that the self-antigen, which became autoantigen, is probably restricted to the beta cell. Preproinsulin, the precursor of insulin, is the only peptide that is uniquely present in beta cells and not in any other cells. In contrast, insulin and C-peptide are secretory products, which leave the beta cells and circulate in blood. In some embodiments, a composition described herein comprises one or more peptide fragments that are present in beta cells and not in any other cells. In some instances, a composition described herein comprises one or more peptide fragments that are not present in circulation (e.g., in a human subject).

In brief, peripheral reintroduction of the primary autoantigen, e.g., preproinsulin peptide fragments in adjuvant, can induce regulatory immune response and reestablish immune tolerance in T1DM patients. If the autoimmune process can be arrested even in this late stage, beta cells can be preserved and possibly permit their regeneration. This is a unique, T1DM-specific, targeted and non-immunosuppressive approach, and is, thus, particularly well-suited for children and young adults with T1DM and for prevention in at-risk human subjects as well.

### Adjuvants

Compositions in accordance with the present disclosure can include an adjuvant that promotes a regulatory immune response (e.g., in a human subject). In some embodiments, the composition includes an adjuvant that comprises an oil and an emulsifier mixed with water. In some embodiments, the composition includes an incomplete Freund's adjuvant (IFA). In some embodiments, the composition can include an alum adjuvant, squalene, killed bacteria, toxoids, inorganic compounds, liposomes, dendrimers, nanoemulsions, and/or the like.

An IFA (commercially available, for example, as Adjuvant Montanide ICA 51 from Seppic Inc., France) typically consists of two components, an oil and an emulsifier. IFAs can be used with antigens to elicit cell-mediated immunity and the production of antibodies of protective isotypes (IgG2a in mice and IgG1 in primates). Different types of adjuvants share similar side effects, such as a reaction at the injection site and pyrogenicity. Alum, a commonly used adjuvant for human vaccine, also may produce an appreciable granulomatous response at the injection site.

The mode of action of an incomplete Freund's adjuvant can involve non-specific as well as specific immune responses (e.g., in a human subject). IFAs can also act as an antigen vehicle and as a slow release or long-term antigen presentation device. This can be an important characteristic of IFA as prolonged peripheral presentation of self-antigens can cause low-avidity auto-reactive T cells to differentiate into memory-like auto regulatory T cells that suppress both auto-reactive CTLs and the antigen presenting cells (APCs) self-antigens presentation. The specific enhancing effect of the IFA on the antigen immunogenicity may lead to increased humoral immunity (e.g., preferentially protective antibody production; IgG1 in humans and IgG2a in mice) and to elicit specific cell-mediated immunity (e.g., Th2 type). Because of the reliability and the duration of protection, the use of autoantigen-specific immunization therapy in T1DM can be advantageous. In some instances, a composition described herein is immunomodulatory. Additionally or alternatively, the composition may not be immunosuppressive. In some instances, a composition described herein elicits a Th2 immune response (e.g., in a human subject to whom the composition is administered). Additionally or alternatively, the composition may not elicit a Th1 immune response (e.g., in a human subject to whom the composition is administered).

### Combination Therapies

Compositions in accordance with the present disclosure can include one or more therapeutics in addition to the one or more preproinsulin peptide fragments described hereinabove. The additional therapeutic can be a therapeutic for T1DM and/or another related or coexisting condition. Examples of such additional therapeutics include, without limitations, proregulatory leukotrienes, cytokines (e.g., IL-10, TGF beta, and the like), or other substances for promoting or enhancing regulatory responses, or restoring self-tolerance. Other examples of additional therapeutics include anti-inflammatory leukotrienes and cytokines (e.g., an IL-1 antagonist) that block autoimmune responses. Further examples of additional therapeutics include agents promoting beta cell regeneration and/or growth (e.g., Exenatide) and/or other anti-inflammatory/anti-autoimmunity agents (e.g., Vitamin D and its analogs).

The one or more additional therapeutics can be part of the composition. Alternatively, the one or more additional therapeutics can be separate to the composition. In some such instances, the one or more additional therapeutics can be administered in combination with the composition. Alternatively, the one or more additional therapeutics can be administered separate to the composition. In some such instances, the one or more additional therapeutics can be administered concurrently with the composition. Alternatively, the one or more additional therapeutics can be administered prior to administration of the composition. For example, the one or more additional therapeutics can be administered about 5 min, 10 min, 15 min, 20 min, 25 min, 30 min, 45 min, 1 h, 1.5 h, 2 h, 2.5 h, 3 h, 3.5 h, 4 h, 4.5 h, 5 h, 5.5 h, 6 h, 7 h, 8 h, 9 h, 10 h, 11h, 12 h, 18 h, 24 h, 30 h, 36 h, 42 h, 48 h, 54 h, 60 h, 72 h, 96 h, 5 days, 6 days, 1 week, 2 weeks, 3 weeks, 4 weeks, 1 month, 2 months, 3 months, 4 months, 5 months, 6 months, or 1 year prior to administration of the composition. Alternatively, the one or more additional therapeutics can be administered subsequent to administration of the composition. For example, the one or more additional therapeutics can be administered about 5 min, 10 min, 15 min, 20 min, 25 min, 30 min, 45 min, 1 h, 1.5 h, 2 h, 2.5 h, 3 h, 3.5 h, 4 h, 4.5 h, 5 h, 5.5 h, 6 h, 7 h, 8 h, 9 h, 10 h, 11h, 12 h, 18 h, 24 h, 30 h, 36 h, 42 h, 48 h, 54 h, 60 h, 72 h, 96 h, 5 days, 6 days, 1 week, 2 weeks, 3 weeks, 4 weeks, 1 month, 2 months, 3 months, 4 months, 5 months, 6 months, or 1 year subsequent to administration of the composition.

### Pharmaceutical Compositions and Kits

In some embodiments, a composition of the present disclosure is formulated as a pharmaceutical composition. In certain instances, a pharmaceutical composition contains a composition of the present disclosure and a pharmaceutically acceptable carrier. For example, a pharmaceutical composition described herein may comprise one or more peptide fragments of preproinsulin in a pharmaceutically acceptable carrier. Alternatively, a pharmaceutical composition described herein may comprise one or more peptide fragments of preproinsulin and one or more additional therapeutics in a pharmaceutically acceptable carrier.

In particular, a pharmaceutical composition described herein may comprise a therapeutically effective amount of one or more peptide fragment of preproinsulin in a pharmaceutically acceptable carrier. Alternatively, a pharmaceutical composition described herein may comprise a therapeutically effective amount of one or more peptide fragments of preproinsulin and one or more additional therapeutics in a pharmaceutically acceptable carrier. In some embodiments, a therapeutically effective amount can be 5 micrograms to 10 milligrams, 0.5 to 4.0 milligrams, or any value there between. In some embodiments, a therapeutically effective amount can be 5, 10, 25, 50, 75, 100, 125, 150, 175, 200, 225, 250, 275, 300, 350, 400, 450, 500, 600, 700, 800, or 900 micrograms, or any value there between. In some embodiments, a therapeutically effective amount can be 1.0, 1.25, 1.5, 1.75, 2.0, 2.25, 2.5, 2.75, 3.0, 3.25, 3.5, 3.75, 4.0, 4.25, 4.5, 4.75, 5.0, 5.5, 6.0, 6.5, 7.0, 7.5, 8.0, 8.5, 9.0, 9.5, or 10 milligrams, or any value there between. Additionally or alternatively, a therapeutically effective amount may be an amount that can elicit a desirable immune response in the subject (e.g., a desirable level of antigen-specific Treg cells, suppression of cytotoxic T cell function, generation of a tolerogenic response, generation of a Th2/Treg response). In further or alternative instances, a therapeutically effective amount is an amount that can achieve at least one clinical endpoint (e.g., improved C-peptide secretion, reduced insulin use, improved HbAlc, closer to normal blood sugar levels, less blood sugar level fluctuation, and the like) in the subject. Additionally or alternatively, a therapeutically effective amount may be an amount that can mitigate at least one symptom of the T1DM (e.g., frequency of hypoglycemia/hyperglycemia, reduced glucosuria, level/number of hospitalization, and level/number of complications such as nephropathy, neuropathy, and retinopathy).

A pharmaceutically acceptable carrier may refer to sterile aqueous or non-aqueous solutions, dispersions, suspensions or emulsions, as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. A composition of the present disclosure or one or more components therein (e.g., the one or more peptide fragment of preproinsulin) can be formulated with pharmaceutically acceptable carriers or diluents as well as any other known adjuvants and excipients in accordance with conventional techniques, such as those disclosed in Remington: The Science and Practice of Pharmacy, 19th Edition, Gennaro, Ed., Mack Publishing Co., Easton, Pa., 1995.

In some embodiments, a pharmaceutical composition disclosed herein may contain one or more peptide fragments of preproinsulin, a pharmaceutically acceptable carrier, and, optionally, one or more additional therapeutics, and adjuvants. A pharmaceutical composition disclosed herein may include those suitable for oral administration, rectal administration, topical administration, inhalation, and parenteral (including subcutaneous, intramuscular, and intra-arterial, intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present disclosure suitable for parenteral administration can be prepared as solutions or suspensions of the active ingredients (e.g., one or more peptide fragments of preproinsulin with or without one or more additional therapeutics) in water. A suitable surfactant can be included, such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for injectable use may include sterile aqueous solutions or dispersions. Furthermore, the compositions can be in the form of sterile powders for the extemporaneous preparation of such sterile injectable solutions or dispersions. In all cases, the final injectable form is preferably sterile and effectively fluid for easy syringability. The pharmaceutical compositions is preferably stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present disclosure can be in a form suitable for topical use, such as, for example, an aerosol, cream, ointment, lotion, dusting powder, mouth washes, gargles, and the like. Further, the compositions can be in a form suitable for use in transdermal devices. These formulations can be prepared, utilizing active ingredients (e.g., one or more peptide fragments of preproinsulin with or without one or more additional therapeutics) disclosed herein, or pharmaceutically acceptable salts thereof, via conventional processing methods. As an example, a cream or ointment is prepared by mixing hydrophilic material and water, together with about 5 wt % to about 10 wt % of the active ingredient, to produce a cream or ointment having a desired consistency.

Pharmaceutical compositions of this disclosure can be in a form suitable for rectal administration, wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations, such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or non-aqueous techniques. A tablet containing a composition of this disclosure can be prepared by compression or molding, optionally with one or more accessory ingredients or adjuvants. Compressed tablets can be prepared by compressing, in a suitable machine, the active ingredient in a free-flowing form, such as powder or granules, optionally mixed with a binder, lubricant, inert diluent, surface active or dispersing agent. Molded tablets can be made by molding in a suitable machine, a mixture of the powdered compound moistened with an inert liquid diluent.

In addition to the aforementioned carrier ingredients, the pharmaceutical formulations described hereinabove can include, as appropriate, one or more additional carrier ingredients, such as diluents, buffers, flavoring agents, binders, surface-active agents, thickeners, lubricants, preservatives (including anti-oxidants), and the like. Furthermore, other adjuvants can be included to render the formulation isotonic with the blood of the intended recipient. Compositions comprising one or more peptide fragments of preproinsulin and optionally, one or more additional therapeutics, can also be prepared in powder or liquid concentrate form.

In some embodiments, unit dosage form for the one or more peptide fragments of preproinsulin and the one or more additional therapeutics are co-formulated. In such embodiments, unit dosage form for the one or more peptide fragment of preproinsulin and unit dosage form for the one or more additional therapeutics may be co-formulated for oral administration, inhalation, topical administration, and/or parenteral administration.

In other embodiments, unit dosage form for the one or more peptide fragments of preproinsulin and unit dosage form for the one or more additional therapeutics are formulated separately. In such embodiments, unit dosage form for the one or more peptide fragments of preproinsulin may be formulated for oral administration and unit dosage form for the one or more additional therapeutics may be formulated for parental administration. Alternatively, unit dosage form for the one or more peptide fragments of preproinsulin may be formulated for parental administration and unit dosage form for the one or more additional therapeutics may be formulated for oral administration. Alternatively, unit dosage form for the one or more peptide fragments of preproinsulin may be formulated for topical administration and unit dosage form for the one or more additional therapeutics may be formulated for parental administration. Alternatively, unit dosage form for the one or more peptide fragments of preproinsulin may be formulated for parental administration and unit dosage form for the one or more additional therapeutics may be formulated for topical administration. Alternatively, unit dosage form for the one or more peptide fragments of preproinsulin may be formulated for oral administration and unit dosage form for the one or more additional therapeutics may be formulated for inhalation. Alternatively, unit dosage form for the one or more peptide fragments of preproinsulin may be formulated for inhalation and unit dosage form for the one or more additional therapeutics may be formulated for oral administration. Alternatively, unit dosage form for the one or more peptide fragments of preproinsulin may be formulated for topical administration and unit dosage form for the one or more additional therapeutics may be formulated for inhalation. Alternatively, unit dosage form for the one or more peptide fragments of preproinsulin may be formulated for inhalation and unit dosage form for the one or more additional therapeutics may be formulated for topical administration.

In some embodiments, a pharmaceutical composition described herein may be formulated to release the one or more peptide fragment of preproinsulin with or without the one or more additional therapeutics immediately upon administration or at any predetermined time period after administration using controlled or extended release formulations. Administration of the pharmaceutical composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window at the site of release; or (iii) a short biological half-life, so that frequent dosing during a day is required in order to sustain a therapeutic level.

Many strategies can be pursued to obtain controlled or extended release in which the rate of release outweighs the rate of metabolism of the pharmaceutical composition. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

The pharmaceutical compositions may be sterilized by conventional sterilization techniques, or may be sterile filtered. The resulting aqueous solutions may be packaged for use as is or lyophilized. The lyophilized preparation may be administered in powder form or combined with a sterile aqueous carrier prior to administration. The pH of the preparations typically will be between 3 and 11, more preferably between 5 and 9 or between 6 and 8, and most preferably between 7 and 8, such as 7 to 7.5. The resulting pharmaceutical compositions in solid form may, for example, be packaged in multiple single-dose units, each containing a fixed amount of one or more peptide fragment of preproinsulin, and, optionally, one or more additional therapeutics, such as in a sealed package of tablets or capsules, or in a suitable dry powder inhaler (DPI) capable of administering one or more doses.

The pharmaceutical compositions can be prepared using standard methods known in the art by mixing the active ingredient (e.g., one or more peptide fragments of preproinsulin, and, optionally, one or more additional therapeutics) having the desired degree of purity with, optionally, pharmaceutically acceptable carriers, excipients, or stabilizers (Remington's Pharmaceutical Sciences (20th edition), ed. A. Gennaro, 2000, Lippincott, Williams & Wilkins, Philadelphia, PA). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides, and other carbohydrates, including glucose, mannose, or dextrin; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the disclosure can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the disclosure can include a pharmaceutically acceptable surfactant at a concentration of 0.005 to 0.02%.

Also provided herein is a kit for treating T1DM autoimmunity including (i) a therapeutically effective amount of a composition in accordance with the present disclosure; and (ii) instructions for administration of the composition to a subject in need thereof.

Additionally, provided herein is a kit for diagnosing and treating T1DM autoimmunity including (i) a T1DM autoimmunity diagnostic (e.g., autoantibody testing-anti-insulin IAA, anti GAD65, anti IA2-insulinoma antigen 2, anti Zn8-zink transporter 8 antibodies, T cell biomarkers, and the like); (ii) a therapeutically effective amount of a composition described hereinabove; and (iii) instructions for diagnosing a subject and administering the composition to the subject if the subject is in need thereof.

### Methods of Manufacture

Also provided herein is a method of making a composition in accordance with the present disclosure for treating T1DM. A person skilled in the art will appreciate that the polypeptide fragments can be synthesized using known polypeptide synthetic methodologies. One illustrative example is provided in Example 1 below.

### Methods of Use

Further provided herein are methods for treating T1DM in a subject in need thereof, wherein administration of a composition comprising one or more preproinsulin peptide fragments to the subject generates or expands autoantigen-specific (e.g., preproinsulin-specific) CD4+ regulatory T (Treg) cells. These cells have the capacity to “home” to the pancreatic beta cells, where they release regulatory cytokines and perform other cell-to-cell regulatory functions. Thus, the methods and compositions described herein can be used to prevent the development or progression of T1DM, or prevent or delay loss of residual beta cell mass, providing a longer remission period and delaying or preventing the onset of usually progressive T1DM-related, complications at a later stage of the life. In addition, the methods described herein can be used to predict whether and/or when a subject, e.g., a subject with ongoing anti-insulin autoimmunity, will progress to T1DM, to evaluate a subject's response to a therapeutic intervention or to determine whether a subject should receive a booster administration of the autoantigen formulation (i.e., a composition containing one or more preproinsulin peptide fragments) or an administration of a cell therapy, as described elsewhere herein.

Therefore, the methods described herein comprise administration (e.g., by intravenous, intramuscular, or subcutaneous routes) of a composition comprising an autoantigen (e.g., preproinsulin, such as one or more peptide fragments of preproinsulin) as described herein to a subject, in an amount sufficient to generate a response that comprises the activation, generation, and/or expansion of Treg cells specific for that autoantigen. As used herein, autoantigen-specific Treg cells may refer to preproinsulin-specific Treg cells, i.e., Treg cells that are specific to the one or more peptide fragments of preproinsulin and/or to one or more epitopes exhibited thereby. In some embodiments, once Treg cells have been stimulated, the methods further comprise harvesting Treg cells (i.e., Treg cell population that comprises autoantigen-specific Treg cells) from the subject, expansion of the cells in vitro, and re-administering the cells to the subject. Administration of a composition of the present disclosure (i.e., a composition containing one or more peptide fragments of preproinsulin) and/or administration of the autoantigen-specific Treg cells to a subject may mitigate autoimmunity to pancreatic β-cells and/or generate a tolerogenic response in the subject.

In some instances, the methods further comprise monitoring the levels and/or function of Treg cells over time. In certain embodiments, the level and/or function of Treg cells is measured 3 hours, 6 hours, 12 hours, 18 hours, 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, or more after administration of the composition of the present disclosure (i.e., a composition containing one or more peptide fragments of preproinsulin) and/or administration of a cell therapy, as described elsewhere herein to the subject. In certain instances, the level and/or function of Treg cells is measured daily, weekly, monthly, or yearly. In some embodiments, the methods further comprise administering (e.g., by intravenous, intramuscular, or subcutaneous routes) one or more (e.g., 1, 2, 3, 4, 5, 6, 7, 8, 9, 10, 11, 12, 13, 14, 15, 16, 17, 18, 19, 20, or more) additional doses, i.e., booster doses of the composition and/or cell therapy to maintain the autoantigen-specific Treg levels and/or function. In some embodiments, the methods further comprise determining a level and/or function of autoantigen-specific Treg cells after administration of the composition and/or cell therapy, as described elsewhere herein, and monitoring the levels and/or function over time to determine when to administer booster doses of the composition and/or cell therapy, e.g., at such time when the Treg levels begin to fall. In some embodiments, booster doses can be administered at predetermined times in order to continue to stimulate Treg cells. In certain instances, booster doses can be administered in intervals of approximately 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, or 6 years. For example, booster doses can be administered in repeated intervals of about 6 months to 2 years. In certain embodiments, the interval can be weekly, monthly, quarterly, semi-annually, or annually. For administering booster dose(s) of the composition, the composition can be configured (i.e., with one or more (e.g., 1, 2, 3, 4, 5, 6, 7, 8, 9, or all) of Peptide 1, Peptide 2, Peptide 3, Peptide 4, Peptide 5, Peptide 6, Peptide 7, Peptide 8, Peptide 9, and Peptide 10 described in Table 1) based on an individual subject and/or select populations of subjects. The selection of peptides (e.g., one or more (e.g., 1, 2, 3, 4, 5, 6, 7, 8, 9, or all) of Peptide 1, Peptide 2, Peptide 3, Peptide 4, Peptide 5, Peptide 6, Peptide 7, Peptide 8, Peptide 9, and Peptide 10 described in Table 1) in booster dose(s) of the composition may be based on a subject-specific profile such that the booster dose is personalized to the individual or population. The selection may be based, for example, on a subject's genotype for one or more genes related to T1DM (e.g., correlated to a subject's antigen-specific autoimmune response to preproinsulin) and/or on a subject's immune response to one or more specific peptides (e.g., as measured by a stimulation assay).

In some embodiments, the induced Treg cells are CD4+. In some embodiments, the Treg cells are also CD25 high, FoxP3+, CD127 low, and/or GITR+. The Treg cells may secrete either or both of IL-10 or TGF-beta in response to stimulation with the preproinsulin peptide fragments. In certain instances, the autoantigen-specific Treg cells are CD4+, CD25 high, CR45RO+, and Foxp3+ cells. In particular instances, the autoantigen-specific Treg cells are CD4+, CD25 high, CR45RO+, Foxp3+, CD127 low and GITR+ cells. In some such instances, the level of Treg cells is monitored by measuring the level of CD4+, CD25 high, CR45RO+, Foxp3+, CD127 low, GITR+ cells, by using methods known in the art, e.g., by flow cytometry, immunofluorescence staining, etc. In certain instances, the function of Treg cells is monitored by measuring a tolerogenic response induced by the Treg cells in the subject. Tolerogenic response may be measured by using methods known in the art, e.g., by flow cytometry, immunofluorescence staining, ELISA, real-time quantitative polymerase chain reaction (RT-qPCR), etc.

In some embodiments, a desirable level of antigen-specific Treg cells is an amount sufficient to suppress cytotoxic T cell function. In some embodiments, a desirable level of antigen-specific Treg cells is an amount sufficient to generate a tolerogenic response in the subject. The tolerogenic response may include increased expression of Treg cells (e.g., CD4+, CD25 high, CR45RO+, Foxp3+ cells, such as CD4+, CD25 high, CR45RO+, Foxp3+, CD127 low, GITR+ cells). In certain instances, the tolerogenic response includes increased expression of CD4+, CD25 high, CR45RO+, Foxp3+ Treg cells that are specific to the one or more peptide fragments of preproinsulin. In particular instances, the tolerogenic response includes increased expression of CD4+, CD25 high, CR45RO+, Foxp3+, CD127 low, GITR+ Treg cells that are specific to the one or more peptide fragments of preproinsulin. Additionally or alternatively, the tolerogenic response may comprise increased expression of one or more anti-inflammatory cytokines. In certain instances, the anti-inflammatory cytokines comprise one or more of interleukin (IL)-1 receptor antagonist, IL-4, IL-6, IL-10, IL-11, IL-13, and transforming growth factor-β (TGF-β). Additionally or alternatively, the tolerogenic response may comprise increased expression of one or more immunomodulatory mediators. In certain instances, the immunomodulatory mediator comprises one or more of programmed death ligand (PDL)-1/-2, cytotoxic T-lymphocyte-associated protein 4 (CTLA-4), and immunoglobulin-like transcript (ILT)-3/4. Additionally or alternatively, the tolerogenic response may comprise increased expression of one or more death receptors, such as one or more of Fas, tumor necrosis factor alpha receptor (TNFαR), DR3, DR4, and DR5. Additionally or alternatively, the tolerogenic response may comprise increased expression of indoleamine 2,3-dioxygenase (IDO) and/or heme oxygenase-1 (HO-1). Additionally or alternatively, the tolerogenic response may comprise decreased expression of one or more pro-inflammatory cytokines. In certain instances, the pro-inflammatory cytokines comprise one or more of IL-1, IL-12, IL-18, tumor necrosis factor alpha (TNF-α), interferon gamma (IFNγ), and granulocyte-macrophage colony stimulating factor (GM-CSF).

In some embodiments, the methods of the present disclosure further comprise the methods described in U.S. Pat. App. Pub. No. 2019/0137483 to Orban, published on May 9, 2019, which is herein incorporated by reference in its entirety. Such methods can be used to predict: whether and/or when a subject, e.g., a subject with ongoing anti-insulin autoimmunity, will progress to T1DM; to evaluate a subject's response to a therapeutic intervention described hereinabove (e.g., therapeutic intervention with a composition containing one or more preproinsulin peptide fragments, or therapeutic intervention comprising a cell therapy, as described elsewhere herein); and/or to determine whether a subject should receive a booster administration of the autoantigen formulation (i.e., a composition containing one or more preproinsulin peptide fragments) or an administration of a cell therapy, as described elsewhere herein. Accordingly, in some embodiments, the methods of the present disclosure further comprise assessing a marker for self-insulin production decline in T1DM.

In certain embodiments, a method described herein further comprises measuring central memory T-cell subpopulation levels and/or measuring the ratio of CD4 naïve T-cell to central memory (CD45RO+CD62L+) T-cell subpopulations for the purpose of: diagnosing T1DM, diagnosing pre-T1DM (e.g., pre-clinical T1DM), diagnosing susceptibility to T1DM, or assessing the effectiveness of a therapeutic intervention described hereinabove (e.g., therapeutic intervention with a composition containing one or more preproinsulin peptide fragments, or therapeutic intervention comprising a cell therapy, as described elsewhere herein). In particular embodiments, central memory T-cells may be defined as CD45RO+CD62L+ T-cells and/or CD4 naïve cells may be defined as CD4+CD45RO-CD62L+ T-cells.

For example, a method for diagnosing T1DM, diagnosing pre-T1DM, or diagnosing susceptibility to T1DM can comprise the steps of: selecting a subject having or suspected of having T1DM, pre-T1DM, or susceptibility to T1DM; extracting a biological sample (e.g., blood, serum, plasma, urine, tissue, cell, etc.) from the subject; optionally determining a level of CD4 naïve (CD45RO-CD62L+) T-cells by immunofluorescence analysis (e.g., by flow cytometry, immunohistochemical staining, etc.) of the sample; determining a level of CD4 central memory (CD45RO+CD62L+) T-cells by immunofluorescence analysis (e.g., by flow cytometry, immunohistochemical staining, etc.) of the sample; and measuring the ratio of CD4 naïve (CD45RO-CD62L+) T-cell to central memory (CD45RO+CD62L+) T-cell subpopulation, and/or the level of CD4 central memory T-cells in the sample, wherein a low/decreasing ratio of CD4 naïve T-cells to CD4 central memory T-cells, or a high/increasing CD4 central memory T-cell level would indicate T1DM, pre-T1DM, or a susceptibility to T1DM. A subject determined (e.g., by the aforementioned method) to have T1DM, pre-T1DM, or susceptibility to T1DM can be selected for treatment with a therapeutic intervention described hereinabove (e.g., therapeutic intervention with a composition comprising one or more preproinsulin peptide fragments, and/or therapeutic intervention comprising a cell therapy, as described elsewhere herein). Alternatively, a high/increasing ratio of CD4 naïve T-cells to CD4 central memory T-cells, or a low/decreasing CD4 central memory T-cell level in the aforementioned method would indicate that the subject does not have T1DM, pre-T1DM, or susceptibility to T1DM. In those instances, such a subject would be determined to be not in need of a therapeutic intervention described herein, at least for the time being. The subject may be reassessed periodically as needed.

Additionally, or alternatively, a method for determining the effectiveness of a therapeutic intervention described herein (e.g., therapeutic intervention with a composition containing one or more preproinsulin peptide fragments, or therapeutic intervention comprising a cell therapy, as described elsewhere herein) can include the steps of: initiating the therapy in a subject (e.g., by administering a composition containing one or more preproinsulin peptide fragments, or administering a cell therapy, as described elsewhere herein); extracting a biological sample (e.g., blood, serum, plasma, urine, tissue, cell, etc.) from the subject; optionally determining a level of CD4 naïve (CD45RO-CD62L+) T-cells by immunofluorescence analysis (e.g., by flow cytometry, immunohistochemical staining, etc.) of the sample; determining a level of CD4 central memory (CD45RO+CD62L+) T-cells by immunofluorescence analysis (e.g., by flow cytometry, immunohistochemical staining, etc.) of the sample; and measuring the ratio of CD4 naïve (CD45RO-CD62L+) T-cell to central memory (CD45RO+CD62L+) T-cell subpopulation, and/or the level of CD4 central memory T-cells in the sample, wherein a high/increasing ratio of CD4 naïve T-cell to central memory T-cell subpopulation, and/or low/decreasing level of CD4 central memory T-cell level during the therapy indicates effectiveness of the therapy. Furthermore, a high/increasing ratio of CD4 naïve T-cell to central memory T-cell subpopulation, and/or low/decreasing level of CD4 central memory T-cells during the therapy may also indicate that the subject would not immediately need a booster dose of the autoantigen formulation (i.e., a composition comprising one or more preproinsulin peptide fragments) and/or a booster dose of a cell therapy, as described elsewhere herein. Alternatively, a low/decreasing ratio of CD4 naïve T-cell to central memory T-cell subpopulation, and/or high/increasing level of CD4 central memory T-cell level during the therapy indicates ineffectiveness of the therapy and/or indicates that the effect of the therapy is decreasing or fading out. Furthermore, a low/decreasing ratio of CD4 naïve T-cell to central memory T-cell subpopulation, and/or high/increasing level of CD4 central memory T-cell level during the therapy may also indicate that the subject would need one or more booster doses of the autoantigen formulation (i.e., a composition comprising one or more preproinsulin peptide fragments), and/or a dose or one or more booster dose of a cell therapy, as described elsewhere herein. In some such instances, 1, 2, 3, 4, 5, 6, 7, 8, 9, 10, 11, 12, 13, 14, 15, 16, 17, 18, 19, 20, or more booster doses can be administered (e.g., by intravenous, intramuscular, or subcutaneous routes). In certain instances, booster doses can be administered in intervals of approximately 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, or 6 years. For example, booster doses can be administered in repeated intervals of about 6 months to 2 years. In certain embodiments, the interval can be weekly, monthly, quarterly, semi-annually, or annually.

Additionally, or alternatively, a method for determining the effectiveness of a therapeutic intervention described herein (e.g., therapeutic intervention with a composition containing one or more preproinsulin peptide fragments, or therapeutic intervention comprising a cell therapy, as described elsewhere herein) can include the steps of: initiating the therapy in a subject (e.g., by administering a composition containing one or more preproinsulin peptide fragments, or administering a cell therapy, as described elsewhere herein); extracting a biological sample (e.g., blood) from the subject; and using the sample to determine pancreatic beta cell function of the subject (e.g., by C-peptide test, such as by average C-peptide plasma concentration (CPAVE) test), wherein a steady pancreatic beta cell function during the therapy indicates effectiveness of the therapy. Furthermore, a steady pancreatic beta cell function during the therapy may also indicate that the subject would not immediately need a booster dose of the autoantigen formulation (i.e., a composition comprising one or more preproinsulin peptide fragments) and/or a booster dose of a cell therapy, as described elsewhere herein. Alternatively, a decline in pancreatic beta cell function during the therapy may indicate ineffectiveness of the therapy and/or may indicate that the effect of the therapy is decreasing or fading out. Furthermore, a decline in pancreatic beta cell function during the therapy may also indicate that the subject would need one or more booster doses of the autoantigen formulation (i.e., a composition comprising one or more preproinsulin peptide fragments), and/or a dose or one or more booster dose of a cell therapy, as described elsewhere herein. In some such instances, 1, 2, 3, 4, 5, 6, 7, 8, 9, 10, 11, 12, 13, 14, 15, 16, 17, 18, 19, 20, or more booster doses can be administered (e.g., by intravenous, intramuscular, or subcutaneous routes). In certain instances, booster doses can be administered in intervals of approximately 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, or 6 years. For example, booster doses can be administered in repeated intervals of about 6 months to 2 years. In certain embodiments, the interval can be weekly, monthly, quarterly, semi-annually, or annually.

For determining effectiveness of a therapeutic intervention (e.g., therapeutic intervention with a composition containing one or more preproinsulin peptide fragments, or therapeutic intervention comprising a cell therapy, as described elsewhere herein) by the method described hereinabove, samples can be extracted from the subject, for example, before the start of therapy (or after the start of therapy but before the onset of changes in cell populations); and/or at approximately 3 hours, 6 hours, 12 hours, 18 hours, 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, or more, of ongoing therapy.

Effectiveness of a therapeutic intervention can be determined by the aforementioned methods at 3 hours, 6 hours, 12 hours, 18 hours, 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, or more after administration of the therapy (i.e., administration of a composition of the present disclosure and/or administration of a cell therapy, as described elsewhere herein) to the subject. In certain instances, the effectiveness of therapy is measured by the aforementioned method daily, weekly, monthly, or yearly.

### Cell Therapy Methods

Described herein are cell therapy methods that can be used to treat, delay or prevent the development or progression of T1DM in a subject. As used herein, cell therapy methods may comprise methods for treating T1DM in a subject by administering to the subject a population of preproinsulin-specific Treg cells. Such preproinsulin-specific Treg cells can be induced, generated and/or expanded by: administration (e.g., by intravenous, intramuscular, or subcutaneous routes) of a composition comprising one or more peptide fragments of preproinsulin to a subject, in an amount sufficient to generate a response that comprises the activation, generation, and/or expansion of Treg cells specific to the one or more peptide fragments of preproinsulin or to one or more epitopes exhibited thereby; harvesting a population of Treg cells from the subject, wherein the population of Treg cells comprises Treg cells specific to the one or more peptide fragments of preproinsulin or to one or more epitopes exhibited thereby; and expansion of the population of Treg cells in vitro or ex vivo, wherein the population of Treg cells comprises Treg cells specific to the one or more peptide fragments of preproinsulin or to one or more epitopes exhibited thereby. In certain embodiments, the expansion comprises further exposing the population of Treg cells to the one or more peptide fragments in vitro. For the purpose of the present disclosure, Treg cells can be expanded ex vivo using combined T cell receptor (TCR)/CD3 stimulation and CD28 in the presence of exogenously added recombinant human IL-2 (rhIL-2) and/or recombinant human IL-15 (rhIL-15), as described, for example, in Peters et al., PloS one 3, e2233 (2008). In certain instances, for ex vivo expansion, a population of Treg cells (e.g., a population of Treg cells comprising Treg cells specific to the one or more peptide fragments of preproinsulin or to one or more epitopes exhibited thereby) can be harvested from the subject, and stimulated with anti-CD3+ anti-CD28 microbeads in the presence of rhIL-2 and/or rhIL-15. For example, for ex vivo expansion, a population of Treg cells can be harvested from the subject, and stimulated with DYNABEADS® (THERMOFISHER SCIENTIFIC) that are conjugated with anti-CD3, anti-CD28, and anti-CD137 antibodies, in the presence of rhIL-2 and/or rhIL-15. For therapy or treatment purpose, such expanded population of Treg cells (e.g., comprising preproinsulin-specific Treg cells) can be re-introduced to the subject as and when need be. For example, if needed, freshly expanded population of Treg cells (e.g., comprising preproinsulin-specific Treg cells) can be re-introduced to the subject. Alternatively, expanded population of Treg cells (e.g., comprising preproinsulin-specific Treg cells) can be stored (e.g., in liquid nitrogen) for future use, and re-introduced to the subject when needed.

As used hereinabove, preproinsulin-specific Treg cells refer to Treg cells that are specific to the one or more peptide fragments of preproinsulin or to one or more epitopes exhibited thereby. In certain embodiments, the stimulated Treg cells can be isolated from a subject, expanded in vitro, and re-introduced (e.g., by intravenous, intramuscular, or subcutaneous routes) into either the same or a different subject. These Treg cells may be naturally occurring in the subject (e.g., expanded from an existing population), but they can also be stimulated or generated by administration of an autoantigen (e.g., preproinsulin), such as by administering to the subject a composition containing one or more peptide fragments of preproinsulin. The methods further comprise monitoring the levels (and, optionally, function) of the induced preproinsulin-specific Treg cells in the subject and re-administering the composition and/or the expanded population of preproinsulin-specific Treg cells of the present disclosure as a booster at a suitable time to further induce preproinsulin-specific Treg cells and to maintain the level and function of such induced T cells so as to affect the progression of the T1DM.

If the Treg cells are isolated from and are to be re-introduced into the same subject (i.e., an autologous transplant), no immune suppression is necessary. If the cells are to be introduced into a different subject (i.e., a heterologous transplant), immune suppression may be recommended, and a good HLA match between the donor and recipient is preferred. If the HLA are substantially matched, the immune system is much less likely to respond adversely. The more HLA proteins that match, the less likely a grafted organ will be rejected by the recipient.

An individual has two of each A, B, Cw, DQ, and DR alleles, where one set of A, B, Cw, DQ, and DR (a “haplotype”) is inherited from each parent. Individuals can be homozygous or heterozygous for the A, B, Cw, DQ, and DR haplotypes. A donor cell is considered to be HLA “matched” or “histocompatible” to an intended recipient, provided the donor cells do not express HLA products that are foreign to the recipient. For example, a donor cell that is homozygous for a haplotype such as HLA-A1, -Cw7, -B8 or HLA-A29, -Cw7, -B8, will match a recipient having a heterozygous HLA profile with both HLA-A1, -Cw7, -B8, and HLA-A29, -Cw8, -B65 haplotypes. See, e.g., Hui et al; Handbook of HLA Typing Techniques, p. 194 (CRC Press, 1993), which is herein incorporated by reference in its entirety.

The transplantation methods described herein can include the steps of isolating preproinsulin-specific Treg cells as described herein, and transferring the cells into a subject, such as a mammal (e.g., a human, such as a T1DM patient). Transplantation can involve, for example, transferring the cells into a subject by injection of a cell suspension into the subject, surgical implantation of a cell mass into a tissue or organ of the subject, or perfusion of a tissue or organ (e.g., the pancreas) with a cell suspension. The route of transferring the cells or transplantation will be determined by the need for the cell to reside in a particular tissue or organ and by the ability of the cell to find and be retained by the desired target tissue or organ. In the case where a transplanted cell is to reside in a particular location, it can be surgically placed into a tissue or organ or simply injected into the bloodstream if the cell has the capability to migrate to the desired target organ.

In some embodiments, the Treg cells described herein are transplanted into a subject who still retains some functional beta cells. For example, the cells can be isolated from a newly-diagnosed patient.

In some embodiments, the Treg cells described herein are transplanted as part of an islet transplantation procedure, to prevent or delay occurrence or reoccurrence of autoimmune destruction of the transplanted cells. Methods for performing such procedures are known in the art, e.g., the Edmonton Protocol. See, e.g., Ryan et al., Diabetes 50:710-719 (2001); Shapiro et al., N. Eng. J. Med. 355:1318-1330 (2006), each of which is herein incorporated by reference in its entirety.

Determination of the appropriate dose of Treg cells is generally made by the clinician, e.g., using parameters or factors known in the art to affect treatment or predicted to affect treatment. Generally, the dose begins with an amount somewhat less than the optimum dose and it is increased by small increments thereafter until the desired or optimum effect is achieved relative to any negative side effects. For example, populations of cells comprising at least about 10⁴, 10⁵, 10⁶, 10⁷, 10⁸, 10⁹, 10¹⁰, or more purified and expanded Treg cells as described herein can be administered, e.g., in one or more doses.

Thus the methods described herein may comprise the steps of isolating one or more preproinsulin-specific Treg cells as described herein, optionally culturing the cell(s) to expand them as needed to obtain a population of preproinsulin-specific Treg cells, and transferring the expanded cells into a subject, such as a mammal (e.g., a human, such as a patient).

### Subjects

In some embodiments, the present disclosure provides a method of treatment for T1DM autoimmunity, including (i) selecting a subject in need of a treatment for T1DM autoimmunity; and (ii) administering a therapeutically effective amount of a composition described herein to the subject. Selection of a patient in need of a treatment can include physical examination by a physician and/or laboratory tests.

A subject described hereinabove can be a mammalian subject, such as a human. In certain instances, the subject is a human patient, such as a subject with T1DM or a subject who is at a risk of developing T1DM. In one embodiment, the subject is a human adult. In another embodiment, the subject is a human juvenile.

In some instances, the subject has T1DM and the treatment achieves at least one clinical endpoint (e.g., improved C-peptide secretion, reduced insulin use, improved HbAlc, closer to normal blood sugar levels, less blood sugar level fluctuation, and the like). Additionally or alternatively, the subject may have T1DM and the treatment may mitigate at least one symptom of the T1DM (e.g., frequency of hypoglycemia/hyperglycemia, reduced glucosuria, level/number of hospitalization(s), and level/number of complications such as nephropathy, neuropathy, and retinopathy).

In some embodiments, the subject has pre-clinical T1DM and the treatment prevents or delays progression to clinical T1DM. For example, compositions and methods of the present disclosure can delay progression of pre-clinical T1DM to clinical T1DM by about 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, or more.

In some embodiments, the subject is predisposed to developing T1DM and the treatment prevents or delays development of T1DM. For example, compositions and methods of the present disclosure can delay development of T1DM by about 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, or more.

Human patients who may be selected for treatment with the methods of the present disclosure can be categorized into the following groups:

(a) Patients with Newly Diagnosed T1DM

Patients in this group generally have approximately 20% residual beta cell function at the time of diagnosis (Staeva-Vieira et al., Clin. and Exp. Immunol., 148:17-31 (2007)) and comprise the group most likely to show a rapid benefit to the composition and methods of the present disclosure. In the U.S., the incidence is 30,000 to 35,000 new T1DM patients annually. As treatment with the composition is expected to be life-long, this pool of patients will, thus, expand annually by at least 30,000 in the U.S. alone, not including the projected 3% annual increase in the incidence of T1DM.

The incidence/100,000 of T1DM in adults is similar to that for children and young adults (ages 1−14=10.3; ages 15-29 years=6.8; ages 30−49=7.3), and many adults are misdiagnosed with type 2 disease due to the misconception of T1DM as a disease only of children (Mobak et al., Diabet Med, 11: 650-655 (1994); Bruno et al., Diabetes Care, 28 (11):2613-2619 (2005)). Using predictive autoantibody markers, a prospective UK study showed that 30% of younger patients diagnosed with type 2 diabetes in fact may have an underlying autoimmune component and usually progress to insulin dependence within 3 years (Turner et al., Lancet 350:1288-1293 (1997); Devendra et al., BMJ 328:750-754 (2004)). This is consistent with the estimate that 10% of persons over age 35 diagnosed with phenotypic type 2 diabetes actually have underlying autoimmune diabetes (Stenstrom et al., Diabetes 54:S68-S72 (2005); Leslie et al., Clinical Rev. 91:1654-1659 (2006)), all of whom are candidates for the T1DM treatment described herein, distinct from those with latent autoimmune diabetes in adults (LADA)). This group of misclassified patients and LADA patients can also be expected to greatly benefit from treatment with the methods described herein, especially since their disease progression takes a little longer to develop. The preproinsulin specific Treg cells induced or activated by the methods described herein, like other antigen-specific Treg cells, can influence effector-autoaggressive T cells of other antigen specificity by so called “infectious tolerance” and or “bystander” effects, which in the case of LADA patients may be particularly beneficial. Correct diagnosis/identification of these patients can be accomplished by methods known in the art, e.g., by serum autoantibody assays performed according to AMA Guidelines (available from Quest Diagnostics and ARUP Labs).

(b) Patients with Established T1DM.

There were an estimated 1.8 million (all age groups) T1DM patients (excluding 10% of patients diagnosed with type 2 diabetes but having underlying LADA) in the U.S in 2003. Although such patients have insufficient insulin production and must be maintained on insulin therapy in the face of an ongoing anti-beta islet cell autoimmune response, some possess measurable levels of beta cells even many years after diagnosis. Importantly, these patients retain the capacity for regenerating functional beta cell activity, and it has been suggested that intervention could enable repletion of beta cells, possibly to physiologically meaningful levels (Staeva-Vieira et al., (2007), supra). In the active disease state, this potential is insufficient to overcome the ongoing loss of beta cells due to the autoimmune response; however, control of the autoimmune attack on beta cells would permit pancreatic beta cell regeneration and concomitant restoration of clinically significant insulin production. As the underlying mechanism of autoimmune destruction of beta cells is the same at all stages of the disease, patients with established T1DM have the potential of benefiting from down-regulation of autoimmune response, as induced by the treatment method described herein.

In addition, the methods described herein can be used in patients who receive a transplant of islet beta cells. Such transplants, without immunosuppression, are unlikely to be successful in the presence of an ongoing autoimmune response against beta cells. In addition, for similar reasons, the methods described herein will be beneficial when used with islet cell regeneration therapies, e.g., administration of exanatide.

(c) Individuals with a High Risk of Developing T1DM.

The average risk of a child developing T1DM is 6% if either of the child's parents or siblings have the disease compared with 0.4% risk in the general population (Tillil and Kobberling, Diabetes, 36:93-99 (1987)). This represents an estimated 360,000 at risk individuals under the age of 15, and 1.3 million at risk individuals for all age groups in the US in 2007. Early intervention has been suggested as a strategy to enhance the probability of successful therapy (Staeva-Vieira et al., (2007), supra). Screening high risk individuals for antibodies to insulin (IAA), glutamic acid decarboxylase (GAD), and insulinoma associated antigen (IA-2A) provides a reliable method of predicting the development of T1DM (Leslie et al., Diabetologia, 42:3-14 (1999); Bingley, Diabetes Care, 24:398 (2001); Achenbach, Curr Diabetes Rep, 5:98-103 (2005)), which can be used to identify candidates for the treatment methods of the present disclosure to prevent, or potentially reverse, autoimmune pathology prior to significant beta cell destruction. Identification of these individuals can be accomplished using methods known in the art, e.g., by serum autoantibody assays performed according to AMA Guidelines (e.g., assays available from Quest Diagnostics and ARUP Labs).

As used herein, “T1DM” also includes LADA (latent autoimmune diabetes in adults), and subjects who can be treated using the methods described herein include those with LADA.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Polypeptide Synthesis

Overlapping preproinsulin 20 amino acid peptide fragments are designed such that each of the peptide fragments overlaps by 10 amino acids with the preceding peptide sequence. These peptides are made as monocomponent HPLC (C18 column) purified peptides, synthesized in a protein-core laboratory on a PROTEIN SYNTHESIZER MODEL 433A from Applied Biosystems, using amino acid preparations from Peptide International. This is a standard solid-phase peptide synthesis (SPPS) procedure, which has the following main steps:

### Chain Assembly

The assembly strategy used in the protein synthesis is ABI (Applies Biosystem Inc.)-Fmoc/Thr. The Fmoc group protects the α-amino group of the amino acid. The peptide is assembled from the C-terminus towards the N-terminus with the α-carboxyl group of the starting amino acid attached to a solid support (resin). The resin used for assembly is polystyrene bead, an insoluble support with size of 400-1000 micron in diameter swelled after washing with NMP (N-methylpyrrolidone). The resin is preloaded with the first amino acid (Thr) from the C-terminus.

The first step in chain assembly is deprotection, or removal of the protecting group. The Fmoc protecting group is removed using 22% piperidine. Conductimetric feedback of carbamate salt formed via removal of Fmoc group with piperidine/NMP can be used to show the coupling efficacy.

After deprotection, the next amino acid is activated and coupled to the deprotected amino end of the growing peptide and forms the peptide bond. Activation of the incoming amino acid carboxyl group is achieved using HBTU/HOBt.

Between couplings, the column is washed with methanol and NMP (N-methylpyrrolidone), which swells the resin and washes out residues. The cycle is repeated until a peptide of a desired length is achieved.

Then the resin is washed with DCM (dichloromethane), which removes NMP from the resin, followed by thoroughly washing the resin with highly volatile methanol, which is an easily removable solvent, and evaporation/drying.

Cleavage from the Resin and Removal of Side Chain Protecting Groups

A cleavage mixture is prepared (0.75 g crystalline phenol+0.25 g ethanedithiol+0.5 ml thioanisol+0.5 ml deionized H2O+10 ml trifluoroaceticacid). The dried peptide-resin is incubated in cool flask in ice bath (10 ml mixture/100-150 mg peptide-resin) for 1.5 h. Then the peptide is isolated from the reaction mixture by glass funnel filtration under high vacuum. The peptide is then precipitated with cold methyl t-butyl ether (MTBE) and vacuum dried.

### Purification Under Sterile Conditions

This step is performed with reverse phase HPLC. Buffer A=0.1% trifluoroaceticacid (TFA) and buffer B=70% acetonitrile, 30% H2O, 0.09% trifluoroaceticacid (TFA). By using C18 column, the elution of the sample is based upon hydrophobicity (hydrophilic sample elute earlier). The peak detection is performed by absorbance measurement of peptide bond at 214 nm and identified by mass spectrometry. The desired fraction is pooled in sterile vials and lyophilized, with a sample taken for AAA (amino acid analysis) analytical rpHPLC and Mass Spectrometry to confirm the sequence.

### Example 2

### Compositions Containing Preproinsulin Peptide Fragments

A. A composition containing preproinsulin peptide fragments is a combination of water-soluble, 20-amino acid long, overlapping, preproinsulin peptide fragments and incomplete Freund's adjuvant solution. The injections/emulsions (the final drug products) are prepared immediately before administration in a lamina-flow protected hood, under sterile condition by using high-pressure sterile syringes as a 50/50 (w/w) emulsion of human preproinsulin peptides mix solution (0.5 ml) by mixing with Montanide ISA51 (0.5 ml) (Seppic Inc.).

B. A composition containing preproinsulin peptide fragments is a combination of water-soluble, 20-amino acid long, overlapping, preproinsulin peptide fragments and incomplete Freund's adjuvant solution. The injections/emulsions (e.g., the final drug product) are pre-prepared (e.g., in a manufacturing setting) and can have an extended expanded shelf life (e.g., years).

C. A composition containing preproinsulin peptide fragments is a combination of water-soluble, 20-amino acid long, overlapping, preproinsulin peptide fragments and incomplete Freund's adjuvant solution. The injections/emulsions (e.g., the final drug products) are prepared as a kit; the two main components (e.g., peptide fragments and adjuvant) in different sealed compartments with a built in mechanism to prepare a fresh mix to be used within short period of time (e.g., days/weeks).

D. A composition containing preproinsulin peptide fragments is a combination of water-soluble, 20-amino acid long, overlapping, preproinsulin peptide fragments and incomplete Freund's adjuvant solution, where incomplete Freund's adjuvant solution is other than Montanide ISA51.

E. A composition containing preproinsulin peptide fragments is a combination of water-soluble, 20-amino acid long, overlapping, preproinsulin peptide fragments and an immunological adjuvant other than incomplete Freund's adjuvant solution (e.g., squalene; killed bacteria and toxoids; aluminum salts-alum/inorganic compounds etc. or liposomes, lipid based nanoparticles, nanoemulsion, nanogels, dendrimers or the like).

### Example 3

Therapy with Preproinsulin Peptide Fragments

A. Administer a composition in accordance with the present disclosure to a subject (e.g., of any age and/or any disease duration) who has been diagnosed with type 1 diabetes mellitus (T1DM) (e.g., a clinical diagnosis, and at least one positive T1DM-specific autoantibodies, such as IAA, GAD65, Ia2, Zn transporter8 or T1DM-specific T cell marker positive).

B. Administer a composition in accordance with the present disclosure to a subject who does not have clinical diagnosis of T1DM, but has at least one positive T1DM-specific autoantibodies (e.g., IAA, GAD65, Ia2, Zn transporter8) or T1DM-specific T cell marker. The subject can have normal glucose status or impaired glucose tolerance tested by oral glucose tolerance test. Such subjects can be identified by family screening of patients with T1DM, or by screening a larger population.

The section headings used herein are for organizational purposes only and are not to be construed as limiting the subject matter described in any way.

While the applicant's teachings are described in conjunction with various embodiments, it is not intended that the applicant's teachings be limited to such embodiments. On the contrary, the applicant's teachings encompass various alternatives, modifications, and equivalents, as will be appreciated by those of skill in the art.

TABLE 3
Sequence Summary Table

| SEQ | | |
|---|---|---|
| ID NO | Description | Sequence (NH2-COOH) |
| 1 | Human preproinsulin | MALWMRLLPLLALLALWGPDPAAAFVNQHL |
| | (NP_000198.1) | CGSHLVEALYLVCGERGFFYTPKTRREAED |
| | | LQVGQVELGGGPGAGSLQPLALEGSLQKRG |
| | | IVEQCCTSICSLYQLENYCN |
| 2 | Peptide 1 | MALWMRLLPLLALLALWGPD |
| 3 | Peptide 2 | LALLALWGPDPAAAFVNQHL |
| 4 | Peptide 3 | PAAAFVNQHLCGSHLVEALY |
| 5 | Peptide 4 | CGSHLVEALYLVCGERGFFY |
| 6 | Peptide 5 | LVCGERGFFYTPKTRREAED |
| 7 | Peptide 6 | TPKTRREAEDLQVGQVELGG |
| 8 | Peptide 7 | LQVGQVELGGGPGAGSLQPL |
| 9 | Peptide 8 | GPGAGSLQPLALEGSLQKRG |
| 10 | Peptide 9 | ALEGSLQKRGIVEQCCTSIC |
| 11 | Peptide 10 | IVEQCCTSICSLYQLENYCN |
| 12 | Preproinsulin - Signal Peptide | MALWMRLLPLLALLALWGPDPAAA |
| 13 | Preproinsulin - B chain | FVNQHLCGSHLVEALYLVCGERGFFYTPKT |
| 14 | Preproinsulin - C peptide | EAEDLQVGQVELGGGPGAGSLQPLALEGSLQ |
| 15 | Preproinsulin - A chain | GIVEQCCTSICSLYQLENYCN |

1. A composition comprising a therapeutically effective amount of one or more peptide fragments of preproinsulin.

2. The composition of claim 1, wherein the one or more peptide fragments span at least 85%, 90%, 95%, or 99% of SEQ ID NO: 1.

3. The composition of claim 2, wherein the one or more peptide fragments span the entire length of SEQ ID NO: 1.

4. The composition of claim 3, wherein the spanned length is uninterrupted.

5. The composition of claim 1, wherein each of the one or more peptide fragments is 10 to 30 amino acids in length.

6. The composition of claim 5, wherein each of the one or more peptide fragments is 20 amino acids in length.

7. The composition of claim 1, wherein each of the one or more peptide fragments comprises an amino acid sequence having at least 85% sequence identity to the amino acid sequence of any one of SEQ ID NOs: 2-11.

8. The composition of claim 7, wherein each of the one or more peptide fragments comprises the amino acid sequence of any one of SEQ ID NOs: 2-11.

9. The composition of claim 1, wherein each of the one or more peptide fragments comprises a preproinsulin epitope.

10. The composition of claim 9, wherein the preproinsulin epitope is not present in insulin.

11-25. (canceled)

26. A method of treating type 1 diabetes mellitus (T1DM) autoimmunity in a subject in need thereof, the method comprising:
(a) administering to the subject the composition of claim 1 in an amount sufficient to generate a response that comprises generation and/or expansion of regulatory T (Treg) cells specific to the one or more peptide fragments in the composition.

27. The method of claim 26, further comprising:
(b) harvesting a population of Treg cells from the subject, wherein the population comprises Treg cells specific to the one or more peptide fragments;
(c) expanding in vitro the population of Treg cells, wherein the population comprises Treg cells specific to the one or more peptide fragments; and/or
(d) administering the expanded population of Treg cells to the subject, wherein the population comprises Treg cells specific to the one or more peptide fragments.

28. The method of claim 27, wherein expanding the population of Treg cells in step (c) comprises exposing the cells to the one or more peptide fragments in vitro.

29. The method of claim 26, further comprising the steps of:
(e) extracting a biological sample from the subject;
(f) measuring a level of CD4 naïve T-cells and/or a level of CD4 central memory T-cells in the sample;
(g) determining the ratio of CD4 naïve T-cell to central memory T-cell subpopulation in the sample; and
(h) administering the composition from step (a) and/or the expanded population of Treg cells from step (d) to the subject if the ratio of CD4 naïve T-cell to central memory T-cell subpopulation is low, and/or if the level of CD4 central memory T-cell is high.

30. The method of claim 29, wherein the CD4 naïve T-cell is CD45RO-CD62L+ and/or the CD4 central memory T-cell is CD45RO+CD62L+.

31. The method of claim 29, wherein the level of CD4 naïve T-cells and/or CD4 central memory T-cells is determined by immunofluorescence analysis of the sample.

32. The method of claim 29, wherein the level of CD4 naïve T-cells and/or level of CD4 central memory T-cells is measured one day, one week, one month, or one year after administration of the composition in step (a) and/or after administration of the expanded population of Treg cells in step (d).

33. The method of claim 29, wherein the level of CD4 naïve T-cells and/or level of CD4 central memory T-cells is measured daily, weekly, monthly, or yearly.

34. The method of claim 29, further comprising the steps of:
(i) extracting a biological sample from the subject;
(j) measuring the level and/or function of Treg cells in the sample; and
(k) administering the composition from step (a) and/or the expanded population of Treg cells from step (d) to the subject if the level and/or function of the Treg cells is low in the sample.

35. The method of claim 34, wherein the Treg cell is a CD4+, CD25 high, CR45RO+, Foxp3+, CD127 low, or GITR+ cell.

36-59. (canceled)
